SEQUENCE LISTING (17 sequences)

SEQ ID NO: 1 - DNA, 269 bp, Synthetic construct
  source 1..269: mol_type = other DNA
agggctctgcgtttgctccaggtagtccgctgctcccttgggcctgggcccactgacagccctggtgcctctggccggctgcacacctcctggcgggcagctgtgtcattgagctccatcatctcttgttctggcaatacctgagagatgaacgggctcaatgacacggaggcctgccctgactgcccacggtgccgtggccaaagaggatctaagggcaccgctgagggcctacctaaccatcgtggggaataaggacagtgtcaccc

SEQ ID NO: 2 - DNA, 269 bp, Synthetic construct
  source 1..269: mol_type = other DNA
agggctctgcgtttgctccaggtagtccgctgctcccttgggcctgggcccactgacagccctggtgcctctggccggctgcacacctcctggcgggcagctgtgttgttttgctgttccaggaagtgttctggcaatacctgcttcctggttcggcaaaacaacacggaggcctgccctgactgcccacggtgccgtggccaaagaggatctaagggcaccgctgagggcctacctaaccatcgtggggaataaggacagtgtcaccc

SEQ ID NO: 3 - DNA, 681 bp, Synthetic construct
  source 1..681: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 4 - DNA, 1744 bp, Synthetic construct
  source 1..1744: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgccaccgcggtggcggccctagagtcgatcgaggaactgaaaaaccagaaagttaactggtaagtttagtctttttgtcttttatttcaggtcccagggctctgcgtttgctccaggtagtccgctgctcccttgggcctgggcccactgacagccctggtgcctctggccggctgcacacctcctggcgggcagctgtgtcattgagctccatcatctcttgttctggcaatacctgagagatgaacgggctcaatgacacggaggcctgccctgactgcccacggtgccgtggccaaagaggatctaagggcaccgctgagggcctacctaaccatcgtggggaataaggacagtgtcacccggatccggtggtggtgcaaatcaaagaactgctcctcagtggatgttgcctttacttctaggcctgtacggaagtgttacttctgctctaaaagctgcggaattgtacccgcggccgatccaccggagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 5 - DNA, 1744 bp, Synthetic construct
  source 1..1744: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgccaccgcggtggcggccctagagtcgatcgaggaactgaaaaaccagaaagttaactggtaagtttagtctttttgtcttttatttcaggtcccagggctctgcgtttgctccaggtagtccgctgctcccttgggcctgggcccactgacagccctggtgcctctggccggctgcacacctcctggcgggcagctgtgttgttttgctgttccaggaagtgttctggcaatacctgcttcctggttcggcaaaacaacacggaggcctgccctgactgcccacggtgccgtggccaaagaggatctaagggcaccgctgagggcctacctaaccatcgtggggaataaggacagtgtcacccggatccggtggtggtgcaaatcaaagaactgctcctcagtggatgttgcctttacttctaggcctgtacggaagtgttacttctgctctaaaagctgcggaattgtacccgcggccgatccaccggagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - DNA, 5501 bp, Homo sapiens
  source 1..5501: mol_type = genomic DNA
agagccagagcaggatggagaggagacgcatcacctccgctgctcgccgctcctacgtctcctcaggggagatgatggtggggggcctggctcctggccgccgtctgggtcctggcacccgcctctccctggctcgaatgccccctccactcccgacccgggtggatttctccctggctggggcactcaatgctggcttcaaggagacccgggccagtgagcgggcagagatgatggagctcaatgaccgctttgccagctacatcgagaaggttcgcttcctggaacagcaaaacaaggcgctggctgctgagctgaaccagctgcgggccaaggagcccaccaagctggcagacgtctaccaggctgagctgcgagagctgcggctgcggctcgatcaactcaccgccaacagcgcccggctggaggttgagagggacaatctggcacaggacctggccactgtgaggcagaagctccaggatgaaaccaacctgaggctggaagccgagaacaacctggctgcctatagacaggaagcagatgaagccaccctggcccgtctggatctggagaggaagattgagtcgctggaggaggagatccggttcttgaggaagatccacgaggaggaggttcgggaactccaggagcagctggcccgacagcaggtccatgtggagcttgacgtggccaagccagacctcaccgcagccctgaaagagatccgcacgcagtatgaggcaatggcgtccagcaacatgcatgaagccgaagagtggtaccgctccaagtttgcagacctgacagacgctgctgcccgcaacgcggagctgctccgccaggccaagcacgaagccaacgactaccggcgccagttgcagtccttgacctgcgacctggagtctctgcgcggcacgaacgagtccctggagaggcagatgcgcgagcaggaggagcggcacgtgcgggaggcggccagttatcaggaggcgctggcgcggctggaggaagaggggcagagcctcaaggacgagatggcccgccacttgcaggagtaccaggacctgctcaatgtcaagctggccctggacatcgagatcgccacctacaggaagctgctagagggcgaggagaaccggatcaccattcccgtgcagaccttctccaacctgcagattcgagaaaccagcctggacaccaagtctgtgtcagaaggccacctcaagaggaacatcgtggtgaagaccgtggagatgcgggatggagaggtcattaaggagtccaagcaggagcacaaggatgtgatgtgaggcaggacccacctggtggcctctgccccgtctcatgaggggcccgagcagaagcaggatagttgctccgcctctgctggcacatttccccagacctgagctccccaccaccccagctgctcccctccctcctctgtccctaggtcagcttgctgccctaggctccgtcagtatcaggcctgccagacggcacccacccagcacccagcaactccaactaacaagaaactcacccccaaggggcagtctggaggggcatggccagcagcttgcgttagaatgaggaggaaggagagaaggggaggagggcggggggcacctactacatcgccctccacatccctgattcctgttgttatggaaactgttgccagagatggaggttctctcggagtatctgggaactgtgcctttgagtttcctcaggctgctggaggaaaactgagactcagacaggaaagggaaggccccacagacaaggtagccctggccagaggcttgttttgtcttttggtttttatgaggtgggatatccctatgctgcctaggctgaccttgaactcctgggctcaagcagtctacccacctcagcctcctgtgtagctgggattatagattggagccaccatgcccagctcagagggttgttctcctagactgaccctgatcagtctaagatgggtggggacgtcctgccacctggggcagtcacctgcccagatcccagaaggacctcctgagcgatgactcaagtgtctcagtccacctgagctgccatccagggatgccatctgtgggcacgctgtgggcaggtgggagcttgattctcagcacttgggggatctgttgtgtacgtggagagggatgaggtgctgggagggatagaggggggctgcctggcccccagctgtgggtacagagaggtcaagcccaggaggactgccccgtgcagactggaggggacgctggtagagatggaggaggaggcaattgggatggcgctaggcatacaagtaggggttgtgggtgaccagttgcacttggcctctggattgtgggaattaaggaagtgactcatcctcttgaagatgctgaaacaggagagaaaggggatgtatccatgggggcagggcatgactttgtcccatttctaaaggcctcttccttgctgtgtcataccaggccgccccagcctctgagcccctgggactgctgcttcttaaccccagtaagccactgccacacgtctgaccctctccaccccatagtgaccggctgcttttccctaagccaagggcctcttgcggtcccttcttactcacacacaaaatgtacccagtattctaggtagtgccctattttacaattgtaaaactgaggcacgagcaaagtgaagacactggctcatattcctgcagcctggaggccgggtgctcagggctgacacgtccaccccagtgcacccactctgctttgactgagcagactggtgagcagactggtgggatctgtgcccagagatgggactgggagggcccacttcagggttctcctctcccctctaaggccgaagaagggtccttccctctccccaagacttggtgtcctttccctccactccttcctgccacctgctgctgctgctgctgctaatcttcagggcactgctgctgcctttagtcgctgaggaaaaataaagacaaatgctgcgcccttccccagagtggactctgatctgttcatgagagggcgggactggggccaagatgtagcctttgacaagaccaactcatttcttattactgatcatctctggggcccatgccctcaccaaattccacccgcagccaaagaggacatacaccagctccctccactcttttcttccttcctctccctgctacctgcaactcaaccagcacaatcttcataggcaagaaagcaaagcagctcaaacatgattcaacactgatcagtgtttaccactggataaatctgagttcacactttccttctctgacctaaatgtgaagtcaggaaacacatgtgccctacttccatcctgagctcagtccccaatctcccaccagcctcaggcccctccacttctcagatcaggtcccagacctgcccatgaaaatggggagcaggctgtaacagatttgtccacatgttcctaccacctgtcccaacccagggtacccacccagagacatctggtatcatttaacaaacacattgaaggacaactggtcttcagagctgaagagagctcctagggggagaagctgggacaacagtgaaataagtagcagcagcaacgacagaagtgaatggtgacaaagactgctgtgatgagcaggtagcctatcagggtgagctccacagccgagcgagtctcaggatctgagaacgaggctgggtagtgcccatgagatgtcacacccagccggaagccagcaactagcacaccctgcctccagcaatagtagatgccccggtcatccagctgggtgaagcggatgtggagctggttgccgtggtcaatgaacaccctcatggacctgttgacacccttcaggtactgtgtgcggtagaggtgctggcggtctttgtcccaggccactgcatgctctggccgggccccaggacaggagatgatgagtccatggcccagtctctgctggtggaactgaatgggcacctggggcacccagggccggctgcccactttggacacatagttaatgatggccagcacgccctcccggatggtctttgtcttctcacagggtactaagcagctccgaaccagcacctcaggcgtgtggtccctggccttggtccgcagcttccttggcacagcccttgagccacaagacaccacatcgggcacggccttgaggtagcgtggggagaggtctgggctctgcaggtagcagaggccgatgcgccactgctccccacgcactccgcagcggtcacagggggtccattcccagaaggtggtgaagacatggaggtgcccatagtattcatctgcaaagggctcctggcccttgtcctggaaagtggccaccattccctcactgttctggatgtccacatcgtaggcgtaaaagtagtcccccttgcgggtgccgcagaagtacaggcctgagtcctcagactgagccctgaaaaccaacaagctgaacatgcggatgctgaagcgggtcagcatgtcgctgcccacacgtacctgggctgcctccgtcagcacccgcccatcaaagtccgtcagcactttggtgtggctgctacctaggtgcttttggtagaaccagactacagctggcacctcttcgggtttgcagtgacagggaagctcaaagctcatgtcggccaggtaggctgcattttcaaacatcaggaaagcagggcagggggtcctctgaaaaatgttttccttctccacaatttcaaaggcctggagcccccatgcccacaggagcacagtggtgagggccaggtgcatacctgaaggaggcaggggtcagaggggcagggcaaaaccagggcattaaaggctcatagggctcctagaaagctctgctaagcggaagcctctagatgaggaaaggattatgcagccaggaaaagcagcaacaatctgcagaggaagccgccaagtgcaaggcaatttattcccagtggatgtacaagatgcccttctaacattccagacctgatctcagggtggggggggaaagccattctagaacctggcctttactcccctttctagaacactggcgctcacccaagaatgggtcaaaggaaaccggaatgagaagggcgggccgaggtgctcgggcagggagatctctgcctcagtgctccaggccctgccctgccagcctggtggaaaagtctttcatcaacctgggggatgaaggaaacccaccctcctgcatatctggccatccgggaggctggctggacctgagctgatggcttgggactttcccaggcccaacctgcacaagaactgagtctctaggggaaaattcaacacctcaaatgatgtagtatttgatcatttgttgattacatgtccattcattggtttggggctataaacattcttgttaagagctgtggagatcagtgtttgtttaccataaagattttgctttttccctttta

SEQ ID NO: 7 - DNA, 1774 bp, Homo sapiens
  source 1..1774: mol_type = genomic DNA
agagccagagcaggatggagaggagacgcatcacctccgctgctcgccgctcctacgtctcctcaggggagatgatggtggggggcctggctcctggccgccgtctgggtcctggcacccgcctctccctggctcgaatgccccctccactcccgacccgggtggatttctccctggctggggcactcaatgctggcttcaaggagacccgggccagtgagcgggcagagatgatggagctcaatgaccgctttgccagctacatcgagaaggttcgcttcctggaacagcaaaacaaggcgctggctgctgagctgaaccagctgcgggccaaggagcccaccaagctggcagacgtctaccaggctgagctgcgagagctgcggctgcggctcgatcaactcaccgccaacagcgcccggctggaggttgagagggacaatctggcacaggacctggccactgtgaggcagaagctccaggatgaaaccaacctgaggctggaagccgagaacaacctggctgcctatagacaggaagcagatgaagccaccctggcccgtctggatctggagaggaagattgagtcgctggaggaggagatccggttcttgaggaagatccacgaggaggaggttcgggaactccaggagcagctggcccgacagcaggtccatgtggagcttgacgtggccaagccagacctcaccgcagccctgaaagagatccgcacgcagtatgaggcaatggcgtccagcaacatgcatgaagccgaagagtggtaccgctccaagtttgcagacctgacagacgctgctgcccgcaacgcggagctgctccgccaggccaagcacgaagccaacgactaccggcgccagttgcagtccttgacctgcgacctggagtctctgcgcggcacgaacgagtccctggagaggcagatgcgcgagcaggaggagcggcacgtgcgggaggcggccagttatcaggaggcgctggcgcggctggaggaagaggggcagagcctcaaggacgagatggcccgccacttgcaggagtaccaggacctgctcaatgtcaagctggccctggacatcgagatcgccacctacaggaagctgctagagggcgaggagaaccggatcaccattcccgtgcagaccttctccaacctgcagattcgagggggcaaaagcaccaaagacggggaaaatcacaaggtcacaagatatctcaaaagcctcacaatacgagttataccaatacaggctcaccagattgtaaatggaacgccgccggctcgcggttagctgcctgcctctcagacacggcgctttgcccagcttgacagggagtgagcctcacccaccccatcctcccaatccccctgagttccctcttcccaggcttcccctaaagggcctggactgcgtcattttcccaggaactgcagtgcccagcccaggacgtggtacagagtaactgtacattaaactggcagagcttgttagtggtaaaggtggtgagtccttgggtgcgcagtggagctgctctggggcctctgagcaagcagcagcctctgtctcacctcttcctgtcactgggagggccccttgggtctcgctgtgcctggacgccaggctctctgctttattctttcatccctgaggctccatcgctcagctcagtgctgactcagttcagaggattcttccctcaggaccgcagctcttgcagtgaataaagttttatgttccctgctcttaatgttaaa

SEQ ID NO: 8 - DNA, 2128 bp, Homo sapiens
  source 1..2128: mol_type = genomic DNA
agagccagagcaggatggagaggagacgcatcacctccgctgctcgccgctcctacgtctcctcaggggagatgatggtggggggcctggctcctggccgccgtctgggtcctggcacccgcctctccctggctcgaatgccccctccactcccgacccgggtggatttctccctggctggggcactcaatgctggcttcaaggagacccgggccagtgagcgggcagagatgatggagctcaatgaccgctttgccagctacatcgagaaggttcgcttcctggaacagcaaaacaaggcgctggctgctgagctgaaccagctgcgggccaaggagcccaccaagctggcagacgtctaccaggctgagctgcgagagctgcggctgcggctcgatcaactcaccgccaacagcgcccggctggaggttgagagggacaatctggcacaggacctggccactgtgaggcagaagctccaggatgaaaccaacctgaggctggaagccgagaacaacctggctgcctatagacaggaagcagatgaagccaccctggcccgtctggatctggagaggaagattgagtcgctggaggaggagatccggttcttgaggaagatccacgaggaggaggttcgggaactccaggagcagctggcccgacagcaggtccatgtggagcttgacgtggccaagccagacctcaccgcagccctgaaagagatccgcacgcagtatgaggcaatggcgtccagcaacatgcatgaagccgaagagtggtaccgctccaagtttgcagacctgacagacgctgctgcccgcaacgcggagctgctccgccaggccaagcacgaagccaacgactaccggcgccagttgcagtccttgacctgcgacctggagtctctgcgcggcacgaacgagtccctggagaggcagatgcgcgagcaggaggagcggcacgtgcgggaggcggccagttatcaggaggcgctggcgcggctggaggaagaggggcagagcctcaaggacgagatggcccgccacttgcaggagtaccaggacctgctcaatgtcaagctggccctggacatcgagatcgccacctacaggaagctgctagagggcgaggagaaccggatcaccattcccgtgcagaccttctccaacctgcagattcgaggtcagtacagcagggcctcgtgggaagggcactggagtcctgccccctcctccagggcctgtaggttgctccagactgggactgaggatcagggcaaagggatccagctctccctgggggccttcgtgacactgcagcgctcctagccagagcctatcataccagggtacttctaggtggggcttgcagctgcccctgtcctgctaggccctggtccctcttcccctccctgcaccccattcgacagcagaactgggtgagagcttgacatctgccctgtctgcagatccctgagcaagcactgcccttctgagtgttttctgtttttgtttttttaactgcttgtcactacagggggcaaaagcaccaaagacggggaaaatcacaaggtcacaagatatctcaaaagcctcacaatacgagttataccaatacaggctcaccagattgtaaatggaacgccgccggctcgcggttagctgcctgcctctcagacacggcgctttgcccagcttgacagggagtgagcctcacccaccccatcctcccaatccccctgagttccctcttcccaggcttcccctaaagggcctggactgcgtcattttcccaggaactgcagtgcccagcccaggacgtggtacagagtaactgtacattaaactggcagagcttgttagtggtaaaggtggtgagtccttgggtgcgcagtggagctgctctggggcctctgagcaagcagcagcctctgtctcacctcttcctgtcactgggagggccccttgggtctcgctgtgcctggacgccaggctctctgctttattctttcatccctgaggctccatcgctcagctcagtgctgactcagttcagaggattcttccctcaggaccgcagctcttgcagtgaataaagttttatgttccctgctcttaatgttaaa

SEQ ID NO: 9 - DNA, 3154 bp, Homo sapiens
  source 1..3154: mol_type = genomic DNA
agagccagagcaggatggagaggagacgcatcacctccgctgctcgccgctcctacgtctcctcaggggagatgatggtggggggcctggctcctggccgccgtctgggtcctggcacccgcctctccctggctcgaatgccccctccactcccgacccgggtggatttctccctggctggggcactcaatgctggcttcaaggagacccgggccagtgagcgggcagagatgatggagctcaatgaccgctttgccagctacatcgagaaggttcgcttcctggaacagcaaaacaaggcgctggctgctgagctgaaccagctgcgggccaaggagcccaccaagctggcagacgtctaccaggctgagctgcgagagctgcggctgcggctcgatcaactcaccgccaacagcgcccggctggaggttgagagggacaatctggcacaggacctggccactgtgaggcagaagctccaggatgaaaccaacctgaggctggaagccgagaacaacctggctgcctatagacaggaagcagatgaagccaccctggcccgtctggatctggagaggaagattgagtcgctggaggaggagatccggttcttgaggaagatccacgaggaggaggttcgggaactccaggagcagctggcccgacagcaggtccatgtggagcttgacgtggccaagccagacctcaccgcagccctgaaagagatccgcacgcagtatgaggcaatggcgtccagcaacatgcatgaagccgaagagtggtaccgctccaagtttgcagacctgacagacgctgctgcccgcaacgcggagctgctccgccaggccaagcacgaagccaacgactaccggcgccagttgcagtccttgacctgcgacctggagtctctgcgcggcacgaacgagtccctggagaggcagatgcgcgagcaggaggagcggcacgtgcgggaggcggccagttatcaggaggcgctggcgcggctggaggaagaggggcagagcctcaaggacgagatggcccgccacttgcaggagtaccaggacctgctcaatgtcaagctggccctggacatcgagatcgccacctacaggaagctgctagagggcgaggagaaccggatcaccattcccgtgcagaccttctccaacctgcagattcgagggggcaaaagcaccaaagacggggaaaatcacaaggtcacaagatatctcaaaagcctcacaatacgagttataccaatacaggctcaccagattgtaaatggaacgccgccggctcgcgaaaccagcctggacaccaagtctgtgtcagaaggccacctcaagaggaacatcgtggtgaagaccgtggagatgcgggatggagaggtcattaaggagtccaagcaggagcacaaggatgtgatgtgaggcaggacccacctggtggcctctgccccgtctcatgaggggcccgagcagaagcaggatagttgctccgcctctgctggcacatttccccagacctgagctccccaccaccccagctgctcccctccctcctctgtccctaggtcagcttgctgccctaggctccgtcagtatcaggcctgccagacggcacccacccagcacccagcaactccaactaacaagaaactcacccccaaggggcagtctggaggggcatggccagcagcttgcgttagaatgaggaggaaggagagaaggggaggagggcggggggcacctactacatcgccctccacatccctgattcctgttgttatggaaactgttgccagagatggaggttctctcggagtatctgggaactgtgcctttgagtttcctcaggctgctggaggaaaactgagactcagacaggaaagggaaggccccacagacaaggtagccctggccagaggcttgttttgtcttttggtttttatgaggtgggatatccctatgctgcctaggctgaccttgaactcctgggctcaagcagtctacccacctcagcctcctgtgtagctgggattatagattggagccaccatgcccagctcagagggttgttctcctagactgaccctgatcagtctaagatgggtggggacgtcctgccacctggggcagtcacctgcccagatcccagaaggacctcctgagcgatgactcaagtgtctcagtccacctgagctgccatccagggatgccatctgtgggcacgctgtgggcaggtgggagcttgattctcagcacttgggggatctgttgtgtacgtggagagggatgaggtgctgggagggatagaggggggctgcctggcccccagctgtgggtacagagaggtcaagcccaggaggactgccccgtgcagactggaggggacgctggtagagatggaggaggaggcaattgggatggcgctaggcatacaagtaggggttgtgggtgaccagttgcacttggcctctggattgtgggaattaaggaagtgactcatcctcttgaagatgctgaaacaggagagaaaggggatgtatccatgggggcagggcatgactttgtcccatttctaaaggcctcttccttgctgtgtcataccaggccgccccagcctctgagcccctgggactgctgcttcttaaccccagtaagccactgccacacgtctgaccctctccaccccatagtgaccggctgcttttccctaagccaagggcctcttgcggtcccttcttactcacacacaaaatgtacccagtattctaggtagtgccctattttacaattgtaaaactgaggcacgagcaaagtgaagacactggctcatattcctgcagcctggaggccgggtgctcagggctgacacgtccaccccagtgcacccactctgctttgactgagcagactggtgagcagactggtgggatctgtgcccagagatgggactgggagggcccacttcagggttctcctctcccctctaaggccgaagaagggtccttccctctccccaagacttggtgtcctttccctccactccttcctgccacctgctgctgctgctgctgctaatcttcagggcactgctgctgcctttagtcgctgaggaaaaataaagacaaatgctgcgcccttcccca

SEQ ID NO: 10 - DNA, 2600 bp, Mus musculus
  source 1..2600: mol_type = genomic DNA
ccaggaagtcaggggcagatttagtccaacccgttcctccataaaggccctgacatcccaggagccagcagaggcagggcaggatggagcggagacgcatcacctctgcgcgccgctcctatgcctccgagacggtggtcaggggcctcggtcctagtcgacaactgggtaccatgccacgcttctccttgtctcgaatgactcctccactccctgccagggtggacttctccctggccggggcgctcaatgctggcttcaaggagacacgggcgagcgagcgtgcagagatgatggagctcaatgaccgctttgctagctacatcgagaaggtccgcttcctggaacagcaaaacaaggcgctggcagctgaactgaaccagcttcgagccaaggagcccaccaaactggctgatgtctaccaggcggagcttcgggagctgcggctgcggctggaccagcttacggccaacagtgcccggctggaggtggagagggacaactttgcacaggacctcggcaccctgaggcagaagctccaagatgaaaccaacctgaggctggaggcagagaacaacctggctgcgtatagacaggaggcagatgaagccaccctggctcgtgtggatttggagagaaaggttgaatcgctggaggaggagatccagttcttaaggaagatctatgaggaggaagttcgagaactccgggagcagctggcccaacagcaggtccacgtggagatggatgtggccaagccagacctcacagcggccctgagagagattcgcactcaatacgaggcagtggccaccagtaacatgcaagagacagaggagtggtatcggtctaagtttgcagacctcacagacgctgcgtcccgcaacgcagagctgctccgccaagccaagcacgaagctaacgactatcgccgccaactgcaggccttgacctgcgatctggagtccctgcgcggcacgaacgagtccctagagcggcaaatgcgcgaacaggaagagcgccatgcgcgggagtcggccagttaccaggaggcacttgctcggctggaggaggagggccaaagcctcaaggaggagatggcccgccacctgcaggagtaccaggatctactcaacgttaagctagccctggacatcgagatcgccacctacaggaaattgctggagggcgaagaaaaccgcatcaccattcctgtacagactttctccaacctccagatccgagggggcaaaagcaccaaagaaggggaaggccaaaaagtcacaagacctctcaaaaggctcacaatacaagttgtcccaatacaggctcaccagattgaaaatggagccctgccagctctcccttagatagatgcgtgctccagctctcccttagatagaggcgtgctccagctctcccttagatagaggcgtgctccagctctcccttagatagaggcgtgctccagctctcccttagatagaggcgtgctccagctctcccttagatagaggcgtgctccagctctcccttagatagatgcgcgcatttcagccacacctttccagcttgtcttcttcctcccaggcctcctctaagggactgaaccatgtcctttgtctagaagcttccaggcccaccctaggtcctggctctgtgtaattaggttataccgatagagctagcctatgctaaaggttaggttgtactaacagagctagcctatgctaaaggttaggttgtactaatagagctagtcttatgctaaaggttaggttgtattaacagagctagcctatgctaaaggttaggttgtactaacagagctagtcatgttaagttaggttgtactaacagagctagcttatgctaaagttaggttgtactaacatagctagcctatgctaaaggtttggttgtactaacagagctggcctatgttaaaggttaggttgtactaatagagctagtcttatgctaaaggttaggttgtattaacagagctaacctatgctaaaggttaggttgtactaacagagctggcctatgttaaaggttaggttgtactaatagagctagcctatgctaaaggttaggttgtactaacagagctggcctatgttaaaggttaggttttactaatagagctagtcttatgctaaaggttaggttgtactaacagagcgagcctatgctaaagattaggttatattaacagagctagcctatgctaaaggttaggttgtactaatagagctagcctatgctaaaggttaggttgtactaatagagctagcctatgctaaaggttaggttgtattaacagagctagccaatgttaaaggcagcaagtccctgggagctccaaggagatactctgaaccctctgagcaaatgcctccggctcaccagtttctgtcgctaggtggtccccttgggtcttgcagtgcctgtgggcaggctctgtgtttgattcatgtgtccccagagttctattgcttcacttcagtgctgattcagcccagagggttagttagttccctctggacggctgctcttgtagtgaataaagctttatgctccctgctcttcatttt

SEQ ID NO: 11 - DNA, 2733 bp, Mus musculus
  source 1..2733: mol_type = genomic DNA
ccaggaagtcaggggcagatttagtccaacccgttcctccataaaggccctgacatcccaggagccagcagaggcagggcaggatggagcggagacgcatcacctctgcgcgccgctcctatgcctccgagacggtggtcaggggcctcggtcctagtcgacaactgggtaccatgccacgcttctccttgtctcgaatgactcctccactccctgccagggtggacttctccctggccggggcgctcaatgctggcttcaaggagacacgggcgagcgagcgtgcagagatgatggagctcaatgaccgctttgctagctacatcgagaaggtccgcttcctggaacagcaaaacaaggcgctggcagctgaactgaaccagcttcgagccaaggagcccaccaaactggctgatgtctaccaggcggagcttcgggagctgcggctgcggctggaccagcttacggccaacagtgcccggctggaggtggagagggacaactttgcacaggacctcggcaccctgaggcagaagctccaagatgaaaccaacctgaggctggaggcagagaacaacctggctgcgtatagacaggaggcagatgaagccaccctggctcgtgtggatttggagagaaaggttgaatcgctggaggaggagatccagttcttaaggaagatctatgaggaggaagttcgagaactccgggagcagctggcccaacagcaggtccacgtggagatggatgtggccaagccagacctcacagcggccctgagagagattcgcactcaatacgaggcagtggccaccagtaacatgcaagagacagaggagtggtatcggtctaagtttgcagacctcacagacgctgcgtcccgcaacgcagagctgctccgccaagccaagcacgaagctaacgactatcgccgccaactgcaggccttgacctgcgatctggagtccctgcgcggcacgaacgagtccctagagcggcaaatgcgcgaacaggaagagcgccatgcgcgggagtcggccagttaccaggaggcacttgctcggctggaggaggagggccaaagcctcaaggaggagatggcccgccacctgcaggagtaccaggatctactcaacgttaagctagccctggacatcgagatcgccacctacaggaaattgctggagggcgaagaaaaccgcatcaccattcctgtacagactttctccaacctccagatccgagaaaccagcctggacaccaaatccgtgtcagaaggccacctcaagaggaacatcgtggtaaagactgtggagatgcgggatggtgaggtcattaaggactcgaagcaggagcacaaggacgtggtgatgtgaggtgtgcccacctggtggcccttgccatgcagtgtgagggcccaaagcttatcctcaaatagtcctgtttgccaggctcagttcccacccacaccagcacttcccttccttctggttttctgcctgtgtgctgcccaaggctcaatcagtgctaagcttcatagatggcatatacccttcaccttcaactaacaggatactcaccccaaaggcgcagtcaggaggggagggaaccccagctgggttagaattggaagggaagaggaaagatgagcagagtagagagatttaacaaatcacttccttcatccttgttgttatggaaaccgttgccagagctggaagtttccacaggctgctggagctagacaacaattcagacagaaagggaaagtccctgaggcaaagtctctctagccagagacctatgcatcccgaatggccactaaggcagtcctgaagggccctccagggtgatgactccagtgtgtcagccccactgagcagctatgcaggttgactgcccacaggcatgtggaaacttggttctcagcacttggcaggatctatggcataagtggagagggaaggtgtactggacggcggagaggagggctccctggcccctaagtgtggatgcagagaggtggagcccaggaagggtctctgcttaggctgcaggggtgccaatggcagaggcactggtagagatcatttggacactggagttgaaagttacaggcaatctgttacacttggctctgaatcctatgaatcaaggaaataacccgttctctggaagacactgaaacaggagagagggacttccgtccactgggcagggtacagatgtgtctcagttgtgaaggtctattcctggctgcacagtccccatccgctcagtcatcttaccctgtgactgctctcagccctgaagaatccacaaccatccttccaaggttctccatccccacaatgactagctgttgctctccaagctaagggaccattccctgtcttatgcatatacgtaatgtcacctatttaggtatcatcctatttgagagtttgaggaactgaaacgtgttgtgttcaagcagcctggtggctagtgccttcatattagagcaccttctctgaggctgattggtgggcaggtagggaagacattgagcagacagtgtccgctcagttgtccttccctcccttccaaggtccctccctctttccaggacatcgcccccccaccccacccctcctttccacctccgctaacctccagagcagtactgtcacctttactcactgggcagaaataaagacatgtgccatagacttccaaaaaaaaaaaaaaaaa

SEQ ID NO: 12 - DNA, 21 bp, synthetic construct
  source 1..21: mol_type = other DNA
tcattgagctccatcatctct

SEQ ID NO: 13 - DNA, 21 bp, Synthetic construct
  source 1..21: mol_type = other DNA
agagatgaacgggctcaatga

SEQ ID NO: 14 - DNA, 21 bp, Synthetic construct
  source 1..21: mol_type = other DNA
ttgttttgctgttccaggaag

SEQ ID NO: 15 - DNA, 21 bp, Synthetic construct
  source 1..21: mol_type = other DNA
cttcctggttcggcaaaacaa

SEQ ID NO: 16 - DNA, 21 bp, Synthetic construct
  source 1..21: mol_type = other DNA
agagatgatggagctcaatga

SEQ ID NO: 17 - DNA, 21 bp, Synthetic construct
  source 1..21: mol_type = other DNA
cttcctggaacagcaaaacaa

(19) United States
(12) Patent Application Publication
(10) Pub. No.: US 2025/0250566 A1
(43) Pub. Date: Aug. 7, 2025

(54) GENE SILENCING BY RECOMBINANT AAV-AMIRNA IN ALEXANDER DISEASE

(71) Applicant: University of Massachusetts, Westborough, MA (US)

(72) Inventors: Jun Xie, Worcester, MA (US); Guangping Gao, Worcester, MA (US); Wassamon Boonying, Worcester, MA (US); Dominic Gessler, Worcester, MA (US)

(73) Assignee: University of Massachusetts, Westborough, MA (US)

(21) Appl. No.: 18/856,127

(22) PCT Filed: Apr. 14, 2023
(86) PCT No.: PCT/US2023/065770

§ 371 Date: Oct. 11, 2024

Related U.S. Application Data
(60) Provisional application No. 63/331,710, filed on Apr. 15, 2022.

(51) Int. Cl. C12N 15/113; A61P 25/28; C12N 15/86

(52) U.S. Cl. CPC C12N 15/113; A61P 25/28; C12N 15/86; C12N 2310/141; C12N 2750/14143; C12N 2750/14145

(57) ABSTRACT

Aspects of the disclosure relate to compositions (e.g., nucleic acids, rAAV vectors, rAAVs, etc.) and methods for treating Alexander disease (AxD). The disclosure is based, in part, on nucleic acids encoding interfering nucleic acids (e.g., artificial microRNAs) that target glial fibrillary acidic protein (GFAP) RNA transcripts. In some embodiments, the interfering nucleic acids are encoded by rAAV vectors. Aspects of the disclosure also provide methods of treating AxD by administering the nucleic acids to a subject.

### RELATED APPLICATIONS

This application is a national stage filing under 35 U.S.C. § 371 of international PCT application PCT/US2023/065770, filed Apr. 14, 2023, which claims priority under 35 U.S.C. § 119(e) to U.S. provisional patent application, U.S. Ser. No. 63/331,710, filed Apr. 15, 2022, the entire contents of each of which are incorporated by reference herein.

### REFERENCE TO AN ELECTRONIC SEQUENCE LISTING

The contents of the electronic sequence listing (U012070176US01-SEQ-KZM.xml; Size: 38,407 bytes; and Date of Creation: Sep. 5, 2024) is herein incorporated by reference in its entirety.

### BACKGROUND

Alexander disease (AxD) is an autosomal dominant neurological disorder affecting approximately one in one million live births. Approximately 95% of AxD patients have been identified by heterozygous mutations in the glial fibrillary acidic protein (GFAP) gene located on the long arm of chromosome 17q21.31. The distinctive feature of AxD are Rosenthal fibers in the cell body region in astrocytes, which are caused by the accumulation of GFAP in the cytoplasm. Currently, there is no therapy available to treat AxD.

### SUMMARY

Aspects of the disclosure relate to compositions (e.g., nucleic acids, rAAV vectors, rAAVs, etc.) and methods for treating Alexander disease (AxD). The disclosure is based, in part, on nucleic acids encoding interfering nucleic acids (e.g., artificial microRNAs) that target glial fibrillary acidic protein (GFAP) RNA transcripts. In some embodiments, the interfering nucleic acids are encoded by rAAV vectors. The inventors have surprisingly discovered that inclusion of an endogenous GFAP promoter in nucleic acids described herein results in astrocyte-specific expression of inhibitory nucleic acids and reduces off-target effects (e.g., cytotoxicity). Aspects of the disclosure also provide methods of treating AxD by administering the nucleic acids or rAAVs described herein to a subject.

Accordingly, in some aspects, the disclosure provides a nucleic acid comprising a nucleic acid sequence encoding an artificial microRNA (amiRNA) that targets a glial fibrillary acidic protein (GFAP) RNA transcript flanked by adeno-associated virus inverted terminal repeats (AAV ITRs). In some embodiments, the AAV ITRs are AAV2 ITRs or a variant thereof.

In some embodiments, an amiRNA comprises: a nucleic acid sequence encoding a pri-miRNA scaffold; a nucleic acid sequence encoding a guide strand; and, a nucleic acid sequence encoding a passenger strand. In some embodiments, a pri-miRNA scaffold is derived from a naturally-occurring pri-miRNA and comprises at least one flanking sequence and a loop-forming sequence comprising at least 4 nucleotides.

In some embodiments, a pri-miRNA scaffold is derived from a pri-miRNA selected from the group consisting of pri-MIR-21, pri-MIR-22, pri-MIR-26a, pri-MIR-30a, pri-MIR-33, pri-MIR-122, pri-MIR-375, pri-MIR-199, pri-MIR-99, pri-MIR-194, pri-MIR-155, and pri-MIR-451.

In some embodiments, a nucleic acid sequence encoding a guide strand and/or a passenger strand comprises a sequence that is at least 70%, 75%, 80%, 85%, 90%, 95%, or 99% identical to the nucleotide sequence set forth in SEQ ID NO: 1 or 2. In some embodiments, a nucleic acid sequence encoding a guide strand and/or a passenger strand comprises or consists of the sequence set forth in SEQ ID NO: 1 or 2.

In some embodiments, a nucleic acid further comprises a promoter operably linked to a nucleic acid sequence encoding the amiRNA. In some embodiments, a promoter comprises a chicken beta actin (CB) promoter or a GFAP promoter. In some embodiments, an endogenous GFAP promoter is a GfaABC1D promoter. In some embodiments, a GfaABC1D promoter comprises the sequence set forth in SEQ ID NO: 3.

In some embodiments, a nucleic acid comprises a self-complementary AAV (scAAV) vector (e.g., as described in U.S. Pat. No. 11,046,955, the entire contents of which are incorporated by reference herein).

In some embodiments, a nucleic acid comprises or consists of the sequence set forth in SEQ ID NO: 4 or 5.

In some aspects, the disclosure provides a recombinant adeno-associated virus (rAAV) comprising a nucleic acid comprising a nucleic acid sequence encoding an artificial microRNA (amiRNA) that targets a glial fibrillary acidic protein (GFAP) RNA transcript flanked by adeno-associated virus inverted terminal repeats; and at least one AAV capsid protein.

In some embodiments, the AAV ITRs are AAV2 ITRs or a variant thereof.

In some embodiments, at least one capsid protein has a serotype selected from an AAV2, AAV3, AAV4, AAV5, AAV6, AAV7, AAV8, AAVrh8, AAV9, AAV10, or AAVrh10 capsid protein. In some embodiments, at least one capsid protein is an AAV9 capsid protein. In some embodiments, an rAAV is a self-complementary AAV (scAAV).

In some aspects, the disclosure provides a method for reducing glial fibrillary acidic protein (GFAP) in a cell or subject, the method comprising administering a nucleic acid or rAAV as described herein to the cell or subject. In some embodiments, the administration reduces GFAP in the brain. In some embodiments, the administration reduces GFAP in the hippocampus and/or olfactory bulbs.

In some aspects, the disclosure provides a method for reducing Rosenthal fiber formation in a subject, the method comprising administering a nucleic acid or rAAV as described herein to the cell or subject. In some embodiments, the administration reduces Rosenthal fiber formation in the brain. In some embodiments, the administration reduces Rosenthal fiber formation in the hippocampus and/or olfactory bulbs.

In some aspects, the disclosure provides a method for treating Alexander disease (AxD) in a subject, the method comprising administering to the subject a nucleic acid or rAAV as described herein.

In some embodiments, a cell or subject is a mammalian cell or mammalian subject. In some embodiments, a cell a mouse, rat, or human cell. In some embodiments, the subject is a mouse, rat, or human subject.

In some embodiments, a cell or subject comprises one or more mutations in a GFAP gene. In some embodiments, or more mutations comprise heterozygous mutations in each copy of a GFAP gene. In some embodiments, a cell or subject has or is suspected of having Alexander disease (AxD).

In some embodiments, administration comprises systemic administration. In some embodiments, administration comprises injection. In some embodiments, injection comprises intravenous injection. In some embodiments, administration results in reduced Rosenthal fiber formation in a subject.

### BRIEF DESCRIPTION OF DRAWINGS

FIGS. 1A-1B show a schematic representation of rAAV vectors for silencing GFAP. FIG. 1A shows amiR-GFAP packaging in an adeno-associated virus serotype 9 (e.g., scAAV9) driven by the ubiquitous chicken beta actin promoter with CMV enhancer (CMVen/CB). FIG. 1B shows amiR-GFAP packaging in an adeno-associated virus serotype 9 (e.g., scAAV9) driven by an endogenous GFAP promoter (GFaABC1D). The rAAV vectors may be self-complementary AAV (scAAV) vectors or single-stranded AAV vectors.

FIGS. 2A-2C show two scAAV9-CMVen/CB-amiR-GFAP vectors down regulated GFAP mRNA and protein in the brain of the GFAP^{+/R236H} mice. FIG. 2A, top panel, shows a decrease of GFAP protein levels from the whole brain after treatment with scAAV9-CMVen/CB-amiR-GFAP vectors for 3 weeks; bottom panel shows quantification of GFAP protein levels. FIG. 2B shows qRT-PCR shows reduction of GFAP mRNA after treatment with scAAV9-CMVen/CB-amiR-GFAP vectors for 3 weeks. FIG. 2C shows GFAP was reduced after treatment with scAAV9-CB6-amiR-GFAP-2 for 3 months (left panel); right panel represents a quantification of western blot.

FIGS. 3A-3B show representative data for body weights growth and survival curves in female (FIG. 3A) and male (FIG. 3B) GFAP mice treated with a scAAV9-CMVen/CB-amiR-GFAP vector.

FIGS. 4A-4B show representative data for body weights and survival curves of female (FIG. 4A) and male (FIG. 4B) GFAP^{+/R236H} mice treated with a scAAV9-CMVen/CB-amiR-GFAP vector; data shown is for a 2-tailed unpaired t-test comparing AAV9 empty as a control.

FIGS. 5A-5B show representative data for scAAV-GFaABC1D-amiR-GFAP vector mediated silencing of GFAP in vivo. FIG. 5A, top panel shows a decreased of GFAP protein levels after treatment with scAAV-GFaABC1D-amiR-GFAP vector in GFAP^{+/R236H} mice for 3 weeks; bottom panel represents quantification of GFAP protein levels. FIG. 5B shows quantitation of GFAP transcript in scAAV-GFaABC1D-amiR-GFAP vector-treated GFAP^{+/R236H} mice for 3 weeks, analysis by qRT-PCR.

FIGS. 6A-6B show representative data for body weights and survival curves of female (FIG. 6A) and male (FIG. 6B) GFAP^{+/R236H} mice treated with scAAV-GFaABC1D-amiR-GFAP vectors; data represents a 2-tailed unpaired t-test comparing AAV9 empty as a control.

FIG. 7 shows representative data for scAAV-GFaABc1D-amiR-GFAP vector mediated silencing of GAFP in vivo. FIG. 7, top panel shows a decreased of GFAP protein levels after treatment with scAAV9-GFaABc1D-amiR-GFAP-2 vector in GFAP^{+/R236H} mice for 3 months; bottom panel represents quantification of GFAP protein levels.

FIG. 8 shows representative fluorescent imaging data for scAAV-GFaABc1D-amiR-GFAP vector mediated silencing of GAFP in the brain. FIG. 8, top left panel shows a decrease in GFAP protein levels in the hippocampus and olfactory bulbs after treatment with scAAV9-GFaABc1D-amiR-GFAP-2 vector in GFAP^{+/R236H} mice for 3 weeks; bottom left panel shows a decrease in GFAP protein levels in the hippocampus and olfactory bulbs after treatment with scAAV9-GFaABc1D-amiR-GFAP-2 vector in GFAP^{+/R236H} mice for 3 months; right panel shows a schematic of the mouse brain identifying the hippocampus and olfactory bulb.

FIG. 9 shows representative images for scAAV-GFaABc1D-amiR-GFAP vector mediated reduction of Rosenthal fibers (Arrows) in the hippocampus and olfactory bulbs after 3 weeks of treatment.

FIG. 10 shows representative images for scAAV-GFaABc1D-amiR-GFAP vector mediated reduction of Rosenthal fibers (Arrows) in the hippocampus and olfactory bulbs after 3 months of treatment.

### DETAILED DESCRIPTION

Aspects of the invention relate to certain interfering RNAs (e.g., miRNAs, such as artificial miRNAs) that when delivered to a subject are effective for reducing the expression of glial fibrillary acidic protein (GFAP) in the subject. In some embodiments, Accordingly, methods and compositions described by the disclosure are useful, in some embodiments, for the treatment of Alexander disease (AxD).

### Isolated Nucleic Acids

In some aspects, the disclosure provides nucleic acid or isolated nucleic acids that are useful for reducing (e.g., inhibiting) expression and/or activity of GFAP. A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The skilled artisan will also realize that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

The isolated nucleic acids of the invention may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more inhibitory RNAs (e.g., miRNAs) comprising a nucleic acid that targets an endogenous mRNA of a subject. The transgene may also comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV5, AAV6,AAV6.2, AAV7, AAV8, AAV9, AAV10, AAV11, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV5, AAV6, AAV6.2, AAV7, AAV8, AAV9, AAV10, AAV11, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR). Without wishing to be bound by any particular theory, a rAAV vector comprising an ITR lacking a functional TRS produces a self-complementary rAAV vector, for example as described by McCarthy (2008) Molecular Therapy 16(10):1648-1656.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., miRNA).

In some embodiments, the GFAP-targeting inhibitory nucleic acids provided herein are small interfering RNAs (siRNA), also known as short interfering RNA or silencing RNA. siRNA, is a class of double-stranded RNA molecules, typically about 20-25 base pairs in length that target nucleic acids (e.g., mRNAs) for degradation via the RNA interference (RNAi) pathway in cells. The specificity of siRNA molecules may be determined by the binding of the guide strand of the molecule to its target RNA. Effective RNAi molecules are generally less than 30 to 35 base pairs in length to prevent the triggering of non-specific RNA interference pathways in the cell via the interferon response, although longer siRNA can also be effective. In some embodiments, the RNAi molecules are 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the RNAi molecules are 8 to 30 base pairs in length, 10 to 15 base pairs in length, 10 to 20 base pairs in length, 15 to 25 base pairs in length, 19 to 21 base pairs in length, or 21 to 23 base pairs in length. In some embodiments, the RNAi molecule is a siRNA, shRNA, or a miRNA. In some embodiments, the RNAi molecule is an artificial microRNA (AmiRNA or amiR).

Following selection of an appropriate target RNA sequence, RNAi (e.g., siRNA, shRNA, miRNA, AmiRNA) molecules that comprise a nucleotide sequence complementary to all or a portion of the target sequence, i.e., an antisense strand or a guide strand, can be designed and prepared using methods known in the art (see, e.g., PCT Publication Number WO 2004/016735; and U.S. Patent Publication Nos. 2004-0077574 and 2008-0081791; each of which is incorporated herein by reference).

The RNAi (e.g., siRNA, shRNA, miRNA, AmiRNA) molecule can be double-stranded (i.e., a dsRNA molecule comprising a guide strand and a complementary passenger strand) or single-stranded (i.e., a ssRNA molecule comprising just a guide strand). The RNAi (e.g., siRNA, shRNA, miRNA, AmiRNA) molecules can comprise a duplex (i.e. comprising annealed sense and guide strands with a 3′ overhang), asymmetric duplex (i.e. a duplex with 3′ and 5′ antisense overhangs), hairpin (i.e. when two regions of the same strand, usually complementary in nucleotide sequence when read in opposite directions, base-pair to form a double helix that ends in an unpaired loop), or asymmetric hairpin (i.e. hairpin with a strand overhang) secondary structure, having self-complementary passenger and guide strands. In some embodiments, the GFAP-targeting inhibitory nucleic acid described herein is an AmiRNA comprising a guide strand (i.e., antisense strand) and a passenger strand (i.e., sense strand). Double-stranded RNAi molecule described herein (e.g., siRNA or miRNA) may comprise RNA strands that are the same length or different lengths. Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary passenger and guide strands, wherein the circular RNA can be processed either in vivo or in vitro to generate an active siRNA molecule capable of mediating RNAi. In some embodiments, small hairpin RNA (shRNA) molecules) are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in the addition or removal of one, two, three, or more nucleotides from the 3′ end and/or (e.g., and) the 5′ end of either or both strands). A spacer can be of a sufficient length to permit the antisense and sense sequences to anneal and form a double-stranded structure (or stem) prior to cleavage of the spacer (and, optionally, subsequent processing steps that may result in the addition or removal of one, two, three, four, or more nucleotides from the 3′ end and/or (e.g., and) the 5′ end of either or both strands). A spacer sequence may be an unrelated nucleotide sequence that is situated between two complementary nucleotide sequence regions which, when annealed into a double-stranded nucleic acid, comprise a shRNA.

In some embodiments, the guide strand of a RNAi molecule described herein (e.g., siRNA, shRNA, miRNA, or AmiRNA targeting GFAP) is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the guide strand is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

In some embodiments, the passenger strand of a RNAi molecule described herein (e.g., siRNA, shRNA, miRNA, AmiRNA targeting GFAP) is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the passenger strand is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

In some aspects, the disclosure provides inhibitory miRNA that specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of GFAP (e.g., human or mouse GFAP). In some embodiments, the inhibitory nucleic acid targets a conserved region in human and mouse GFAP. As used herein “continuous bases” refers to two or more nucleotide bases that are covalently bound (e.g., by one or more phosphodiester bond, etc.) to each other (e.g., as part of a nucleic acid molecule). In some embodiments, the at least one miRNA is about 50%, about 60% about 70% about 80% about 90%, about 95%, about 99% or about 100% identical to the two or more (e.g., 2, 3, 4, 5, 6, 7, 8, 9, 10, 11, 12, 13, 14, 15, 16, 17, 18, 19, 20, 21, 22, 23, 24, 25, or more) continuous nucleotide bases of human GFAP NCBI sequences NM_002055.5, NM_001131019.3, NM_001242376.3, or NM_001363846.2, SEQ ID NO: 6-9. In some embodiments, the inhibitory RNA is a miRNA which comprises or is encoded by the sequence set forth in any one of SEQ ID NOs: 1 or 2.

In some embodiments, the at least one miRNA is about 50%, about 60% about 70% about 80% about 90%, about 95%, about 99% or about 100% identical to the two or more (e.g., 2, 3, 4, 5, 6, 7, 8, 9, 10, 11, 12, 13, 14, 15, 16, 17, 18, 19, 20, 21, 22, 23, 24, 25, or more) continuous nucleotide bases of mouse GFAP NCBI sequences NM_001131020.1 or NM_010277.3 SEQ ID NO: 10-11. In some embodiments, the inhibitory RNA is a miRNA which comprises or is encoded by the sequence set forth in any one of SEQ ID NOs: 1 or 2.

In some embodiments, a RNAi molecule described herein (e.g., siRNA, shRNA, miRNA, AmiRNA) comprises a guide strand comprising a region of complementarity to a target region in a GFAP mRNA. In some embodiments, the region of complementarity is at least 80%, at least 85%, at least 90%, at least 91%, at least 92%, at least 93%, at least 94%, at least 95%, at least 96%, at least 97%, at least 98%, at least 99%, or 100% complementary to a target region in a GFAP mRNA. In some embodiments, the target region is a region of consecutive nucleotides in the GFAP mRNA. In some embodiments, a complementary nucleotide sequence need not be 100% complementary to that of its target to be specifically hybridizable or specific for GFAP mRNA.

In some embodiments, a RNAi molecule described herein (e.g., siRNA, shRNA, miRNA, AmiRNA) comprises a guide strand that comprises a region of complementarity to an GFAP mRNA sequence and the region of complementarity is in the range of 8 to 15, 8 to 30, 8 to 40, or 10 to 50, or 5 to 50, or 5 to 40 nucleotides in length. In some embodiments, the region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of an GFAP mRNA (e.g., GFAP coding sequence set forth in any one of SEQ ID NOs: 6-11). In some embodiments, the region of complementarity comprises a nucleotide sequence that contains no more than 1, 2, 3, 4, or 5 base mismatches compared to the complementary portion of a GFAP mRNA (e.g., GFAP coding sequence set forth in any one of SEQ ID NOs: 6-11). In some embodiments, the region of complementarity comprises a nucleotide sequence that has up to 3 mismatches over 15 bases, up to 2 mismatches over 10 bases, or up to 1 mismatch over 5 bases.

In some embodiments, a RNAi molecule described herein (e.g., siRNA, shRNA, miRNA, AmiRNA) comprises a guide strand comprising a nucleotide sequence that is complementary (e.g., at least 85%, at least 90%, at least 95%, or 100%) to a target sequence as set forth in any one of SEQ ID NOs: 6-11. In some embodiments, a RNAi molecule described herein (e.g., siRNA, shRNA, miRNA, AmiRNA) comprises a guide strand comprising a nucleotide sequence that is complementary (e.g., at least 85%, at least 90%, at least 95%, or 100%) to a target sequence as set forth in SEQ ID NOs: 16 or 17. In some embodiments, siRNA molecules comprise a guide strand comprising a nucleotide sequence that is at least 85%, at least 90%, at least 95%, or 100% identical to the sequence as set forth in SEQ ID NO: 12 or 14. In some embodiments, siRNA molecules comprise a guide strand comprising at least 6, at least 7,at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequence as set forth in SEQ ID NO: 12 or 14.

In some embodiments, the GFAP-targeting RNAi molecule described herein (e.g., siRNA, shRNA, miRNA, AmiRNA) comprises a guide strand that is 18-25 nucleosides (e.g., 18, 19, 20, 21, 22, 23, 24, or 25 nucleosides) in length and comprises a region of complementarity to the sequence as set forth in any one of SEQ ID NOs: 6-11, wherein the region of complementarity is at least 13 nucleotides (e.g., 13, 14, 15, 16, 17, 18, or 19 nucleotides) in length. In some embodiments, the region of complementarity is fully complementarity with all or a portion of its target sequence. In some embodiments, the region of complementarity includes 1, 2, 3, or more mismatches.

In some embodiments, the GFAP-targeting RNAi molecule described herein (e.g., siRNA, shRNA, miRNA, AmiRNA) comprises a guide strand that comprises at least 15 consecutive nucleosides of (e.g., at least 15, at least 16, at least 17, at least 18, at least 19, or at least 20) the sequence of any one of SEQ ID NO: 12 or 14. In some embodiments, the GFAP-targeting siRNA or shRNA further comprises a passenger strand that comprises at least 15 consecutive nucleosides (e.g., at least 15, at least 16, at least 17, at least 18, at least 19, or at least 20) complementary to the sequence of any one of SEQ ID NO: 13 or 15.

In some embodiments, the GFAP-targeting RNAi molecule described herein is an AmiRNA comprises a nucleic acid sequence at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1 or 2.

The overall length of a RNAi molecule described herein (e.g., siRNA, shRNA, miRNA, AmiRNA) can vary from about 14 to about 100 nucleotides depending on the type of siRNA molecule being designed. Generally, between about 14 and about 50 of these nucleotides are complementary to the RNA target sequence, i.e., constitute the specific antisense sequence of the RNAi molecule described herein (e.g., siRNA, shRNA, miRNA, AmiRNA). For example, when the siRNA is a double- or single-stranded a RNAi molecule described herein (e.g., siRNA, miRNA), the length can vary from about 14 to about 50 nucleotides, whereas when the siRNA is a shRNA, AmiRNA, or a circular molecule, the length can vary from about 40 nucleotides to about 100 nucleotides.

An RNAi molecule described herein (e.g., siRNA, shRNA, miRNA, AmiRNA) comprises may comprise a 3′ overhang at one end of the molecule, the other end may be blunt-ended or have also an overhang (5′ or 3′). When a RNAi molecule described herein (e.g., siRNA, shRNA, miRNA, AmiRNA) comprises an overhang at both ends of the molecule, the length of the overhangs may be the same or different. In one embodiment, a RNAi molecule described herein (e.g., siRNA, shRNA, miRNA, AmiRNA) comprises 3′ overhangs of about 1 to about 3 nucleotides on both ends of the molecule. In some embodiments, a RNAi molecule described herein (e.g., siRNA, shRNA, miRNA, AmiRNA) comprises 3′ overhangs of about 1 to about 3 nucleotides on the passenger strand. In some embodiments, a RNAi molecule described herein (e.g., siRNA, shRNA, miRNA, AmiRNA) comprises 3′ overhangs of about 1 to about 3 nucleotides on the guide strand. In some embodiments, a RNAi molecule described herein (e.g., siRNA, shRNA, miRNA, AmiRNA) comprises 3′ overhangs of about 1 to about 3 nucleotides on both the passenger strand and the guide strand.

It should be appreciated that an isolated nucleic acid or vector (e.g., rAAV vector), in some embodiments comprises a nucleic acid sequence encoding more than one (e.g., a plurality, such as 2, 3, 4, 5, 10, or more) miRNAs. In some embodiments, each of the more than one miRNAs targets (e.g., hybridizes or binds specifically to) the same target gene (e.g., an isolated nucleic acid encoding three unique miRNAs, where each miRNA targets the GFAP gene). In some embodiments, each of the more than one miRNAs targets (e.g., hybridizes or binds specifically to) a different target gene.

In some aspects, the disclosure provides isolated nucleic acids and vectors (e.g., rAAV vectors) that encode one or more artificial miRNAs. As used herein “artificial miRNA”, “amiRNA”, or “amiR” refers to an endogenous pri-miRNA or pre-miRNA (e.g., a miRNA backbone, which is a precursor miRNA capable of producing a functional mature miRNA), in which the miRNA and miRNA* (e.g., passenger strand of the miRNA duplex) sequences have been replaced with corresponding amiRNA/amiRNA* sequences that direct highly efficient RNA silencing of the targeted gene, for example as described by Eamens et al. (2014), Methods Mol. Biol. 1062:211-224. For example, in some embodiments an artificial miRNA comprises a miR-155 pri-miRNA backbone into which a sequence encoding a mature GFAP miRNA has been inserted in place of the endogenous miR-155 mature miRNA-encoding sequence. In some embodiments, miRNA (e.g., an artificial miRNA targeting GFAP) as described by the disclosure comprises a miR-155 backbone sequence, a miR-30 backbone sequence, a mir-64 backbone sequence, a miR-122 backbone sequence, a pri-MIR-21, a pri-MIR-22, a pri-MIR-26a, a pri-MIR-30a, a pri-MIR-33, a pri-MIR-122, a pri-MIR-375, a pri-MIR-199, a pri-MIR-99, a pri-MIR-194, a pri-MIR-155, or a pri-MIR-451.

A region comprising a transgene (e.g., a second region, third region, fourth region, etc.) may be positioned at any suitable location of the isolated nucleic acid. The region may be positioned in any untranslated portion of the nucleic acid, including, for example, an intron, a 5′ or 3′ untranslated region, etc.

In some cases, it may be desirable to position the region (e.g., the second region, third region, fourth region, etc.) upstream of the first codon of a nucleic acid sequence encoding a protein (e.g., a protein coding sequence). For example, the region may be positioned between the first codon of a protein coding sequence) and 2000 nucleotides upstream of the first codon. The region may be positioned between the first codon of a protein coding sequence and 1000 nucleotides upstream of the first codon. The region may be positioned between the first codon of a protein coding sequence and 500 nucleotides upstream of the first codon. The region may be positioned between the first codon of a protein coding sequence and 250 nucleotides upstream of the first codon. The region may be positioned between the first codon of a protein coding sequence and 150 nucleotides upstream of the first codon.

In some cases (e.g., when a transgene lacks a protein coding sequence), it may be desirable to position the region (e.g., the second region, third region, fourth region, etc.) upstream of the poly-A tail of a transgene. For example, the region may be positioned between the first base of the poly-A tail and 2000 nucleotides upstream of the first base. The region may be positioned between the first base of the poly-A tail and 1000 nucleotides upstream of the first base. The region may be positioned between the first base of the poly-A tail and 500 nucleotides upstream of the first base. The region may be positioned between the first base of the poly-A tail and 250 nucleotides upstream of the first base. The region may be positioned between the first base of the poly-A tail and 150 nucleotides upstream of the first base. The region may be positioned between the first base of the poly-A tail and 100 nucleotides upstream of the first base. The region may be positioned between the first base of the poly-A tail and 50 nucleotides upstream of the first base. The region may be positioned between the first base of the poly-A tail and 20 nucleotides upstream of the first base. In some embodiments, the region is positioned between the last nucleotide base of a promoter sequence and the first nucleotide base of a poly-A tail sequence.

In some cases, the region may be positioned downstream of the last base of the poly-A tail of a transgene. The region may be between the last base of the poly-A tail and a position 2000 nucleotides downstream of the last base. The region may be between the last base of the poly-A tail and a position 1000 nucleotides downstream of the last base. The region may be between the last base of the poly-A tail and a position 500 nucleotides downstream of the last base. The region may be between the last base of the poly-A tail and a position 250 nucleotides downstream of the last base. The region may be between the last base of the poly-A tail and a position 150 nucleotides downstream of the last base.

It should be appreciated that in cases where a transgene encodes more than one miRNA, each miRNA may be positioned in any suitable location within the transgene. For example, a nucleic acid encoding a first miRNA may be positioned in an intron of the transgene and a nucleic acid sequence encoding a second miRNA may be positioned in another untranslated region (e.g., between the last codon of a protein coding sequence and the first base of the poly-A tail of the transgene).

In some embodiments, the transgene further comprises a nucleic acid sequence encoding one or more expression control sequences (e.g., a promoter, etc.). Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459). The cleavage activity of the 2A sequence has previously been demonstrated in artificial systems including plasmids and gene therapy vectors (AAV and retroviruses) (Ryan, M D et al., EMBO, 1994; 4:928-933; Mattion, N M et al., J Virology, November 1996; p. 8124-8127; Furler, S et al., Gene Therapy, 2001; 8:864-873;

and Halpin, C et al., The Plant Journal, 1999; 4:453-459; de Felipe, P et al., Gene Therapy, 1999; 6:198-208; de Felipe, P et al., Human Gene Therapy, 2000; 11:1921-1931.; and Klump, H et al., Gene Therapy, 2001; 8:811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is an chicken β-actin promoter. In some embodiments, a promoter is an enhanced chicken-actin promoter. In some embodiments, a promoter is a U6 promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression. In some embodiments the native promoter is a GFAP promoter. In some embodiments the GAFP promotor is a GfaABC1D promoter.

In some embodiments, GfaABC1D is a promotor that selectively targets astrocytes for example as described in, e.g., Griffin, J. M., Fackelmeier, B., Fong, D. M. et al. Astrocyte-selective AAV gene therapy through the endogenous GFAP promoter results in robust transduction in the rat spinal cord following injury. Gene Ther 26, 198-210 (2019). In some embodiments the promoter is a GfaABC1D promoter. In some embodiments, the nucleic acid sequence of a GfaABC1D promoter is set forth in SEQ ID NO: 3. In some embodiments, the nucleic acid encoding an artificial microRNA (amiRNA or amiRNA) targeting GFAP comprises a promoter that is at least 60%, at least 65% at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 3.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: an astrocyte specific promoter (GfaABC1D), a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan. In some embodiments, the tissue specific promoter is an astrocyte specific promotor. In some embodiments, the astrocyte specific promoter is a GfaABC1D promoter.

Aspects of the disclosure relate to an isolated nucleic acid comprising more than one promoter (e.g., 2, 3, 4, 5, or more promoters). For example, in the context of a construct having a transgene comprising a first region encoding a protein and an second region encoding an inhibitory RNA (e.g., AmiRNA targeting GFAP), it may be desirable to drive expression of the protein coding region using a first promoter sequence (e.g., a first promoter sequence operably linked to the protein coding region), and to drive expression of the inhibitory RNA encoding region with a second promoter sequence (e.g., a second promoter sequence operably linked to the inhibitory RNA encoding region). Generally, the first promoter sequence and the second promoter sequence can be the same promoter sequence or different promoter sequences. In some embodiments, the first promoter sequence (e.g., the promoter driving expression of the protein coding region) is a RNA polymerase III (polIII) promoter sequence. Non-limiting examples of polIII promoter sequences include U6 and H1 promoter sequences. In some embodiments, the second promoter sequence (e.g., the promoter sequence driving expression of the inhibitory RNA) is a RNA polymerase II (polII) promoter sequence. Non-limiting examples of polII promoter sequences include T7, T3, SP6, RSV, and cytomegalovirus promoter sequences. In some embodiments, a polIII promoter sequence drives expression of an inhibitory RNA (e.g., miRNA) encoding region. In some embodiments, a polII promoter sequence drives expression of a protein coding region.

Reporter sequences (e.g., nucleic acid sequences encoding a reporter protein) that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of a nucleic acid.

### Recombinant Adeno-Associated Viruses (rAAVs)

In some aspects, the disclosure provides isolated AAVs. As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV2, AAV3, AAV4, AAV5, AAV6, AAV8, AAVrh8, AAV9, and AAV10. In some embodiments, an AAV capsid protein is of an AAV9 serotype.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the instant disclosure relates to a composition comprising the host cell described above. In some embodiments, the composition comprising the host cell above further comprises a cryopreservative.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural. accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., guide RNA) from a transcribed gene. The foregoing methods for packaging recombinant vectors in desired AAV capsids to produce the rAAVs of the disclosure are not meant to be limiting and other suitable methods will be apparent to the skilled artisan.

In some embodiments, any one or more thymidine (T) nucleotides or uridine (U) nucleotides in a sequence provided herein, including a sequence provided in the sequence listing, may be replaced with any other nucleotide suitable for base pairing (e.g., via a Watson-Crick base pair) with an adenosine nucleotide. For example, in some embodiments, any one or more thymidine (T) nucleotides in a sequence provided herein, including a sequence provided in the sequence listing, may be suitably replaced with a uridine (U) nucleotide or vice versa.

### Methods for Treating Alexander Disease

Methods for delivering a transgene (e.g., an inhibitory RNA, such as a miRNA) to a subject are provided by the disclosure. The methods typically involve administering to a subject an effective amount of an isolated nucleic acid encoding an interfering RNA capable of reducing expression of glial fibrillary acidic protein (GFAP), or a rAAV comprising a nucleic acid described herein for expressing an inhibitory RNA capable of reducing expression and/or activity of GFAP. In some embodiments, administration of the nucleic acid or the rAAV described herein reduces GFAP in the brain. In some embodiments, administration of the nucleic acid or the rAAV described herein reduces GFAP activity in the brain. In some embodiments, administration of the nucleic acid or the rAAV described herein reduces GFAP expression and/or activity in the hippocampus and/or olfactory bulbs. In some embodiments, the method typically involved administering to a subject an effective amount of a nucleic acid encoding an interfering RNA targeting GFAP or a rAAV thereof, which results in reduced level of Rosenthal fibers. In some embodiments, administration of the nucleic acid or the rAAv reduces Rosenthal fibers in the brain. In some embodiments, administration of the nucleic acid or the rAAv reduces Rosenthal fibers in the hippocampus and/or olfactory bulbs.

As used herein. “Alexanders disease” or “AxD”, is an autosomal dominant neurological disorder, and a type of leukodystrophy. Leukodystrophy is a neurological condition caused by abnormalities in the myelin, which insulates protects nerve fibers in your brain. AxD affects approximately one in one million live births. Approximately 95% of AxD patients have been identified by heterozygous mutations in the glial fibrillary acidic protein (GFAP) gene located on the long arm of chromosome 17q.31.

The distinctive features of AxD show the Rosenthal fibers in the cell body region of astrocytes, caused by the accumulation of GFAP in the cytoplasm and the destruction of myelin. Rosenthal fibers are abnormal clumps of protein that accumulate in astrocytes, which impair cytoskeleton formation and astrocyte survival and function leading to demyelination and destruction of the white matter in the brain. Myelin enables the electrical impulses in the brain to transmit efficiently.

GFAP is a type III intermediate filament protein, expressed in astrocytes, as well as many other cells in the central nervous system (CNS). GFAP concentration is different depending on the brain region, with the highest levels found in the hippocampus, olfactory bulb, medulla oblongata, and cervical spinal cord. GFAP plays a role in cell communication in the brain, proper functioning of the blood brain barrier, and maintaining astrocyte mechanical strength and cell shape. Under normal conditions, GFAP proteins form filaments that support the nervous system, however when overproduced such as in AxD, GFAP kills cells and damages myelin. Although the disease can be inherited, most cases are caused by sporadic mutations. AxD is most common in infancy or early childhood, although it has been found to occur at any age. There is no cure for AxD, and it is often fatal.

In some embodiments, the subject has Rosenthal fibers in the hippocampus and/or olfactory bulb, e.g., as described in Hagemann et al., Alexander disease-associated glial fibrillary acidic protein mutations in mice induce Rosenthal fiber formation and a white matter stress response. J Neurosci. 2006; 26(43):11162-11173. In some embodiments, the treatment disclosed herein is effective for reducing the formation of Rosenthal fibers. In some embodiments, the treatment disclosed herein is effective for reducing the formation of Rosenthal fibers in the hippocampus and/or olfactory bulb.

In some embodiments, the subject has increased levels of GFAP in the hippocampus and/or olfactory bulb (see, e.g., Jany et al., GFAP Expression as an Indicator of Disease Severity in Mouse Models of Alexander Disease. ASN Neuro. 2013; 5(2)). In some embodiments, the treatment disclosed herein is effective for reducing the expression and/or activity of GFAP. In some embodiments, the treatment disclosed herein is effective for reducing the expression and/or activity of GFAP in the hippocampus and/or olfactory bulb.

An “effective amount” of a substance is an amount sufficient to produce a desired effect. In some embodiments, an effective amount of an isolated nucleic acid is an amount sufficient to transfect (or infect in the context of rAAV mediated delivery) a sufficient number of target cells of a target tissue of a subject. In some embodiments, a target tissue is central nervous system (CNS) tissue (e.g., brain tissue, spinal cord tissue, cerebrospinal fluid (CSF), etc.). In some embodiments, an effective amount of an isolated nucleic acid (e.g., which may be delivered via an rAAV) may be an amount sufficient to have a therapeutic benefit in a subject, e.g., to reduce the expression of a pathogenic gene or protein (e.g., GFAP), to reduce the activity of a pathogenic protein (e.g., GFAP) to extend the lifespan of a subject, to improve in the subject one or more symptoms of disease (e.g., a symptom of Alexander disease), etc. The effective amount will depend on a variety of factors such as, for example, the species, age, weight, health of the subject, and the tissue to be targeted, and may thus vary among subject and tissue as described elsewhere in the disclosure.

As used herein, the term “treating”, “treat”, or “treatment” refers to the application or administration of a composition (e.g., an isolated nucleic acid or rAAV as described herein) to a subject, who has a disease or disorder associated with increased expression of GFAP and/or increased levels of Rosenthal fibers (including leukodystrophy (e.g., Alexander disorder), with the purpose to cure, heal, alleviate, relieve, alter, remedy, ameliorate, improve, or affect the disorder, the symptom of the disease, or the predisposition toward a disease associated with genetic mutations in the gene for GFAP, increased GFAP expression, and increased formation of Rosenthal fibers. In some embodiments, the subject has a mutation in the GFAP gene. In some embodiments the subject has increased expression of GFAP relative to a subject that does not have AxD. In some embodiments, the subject has increased formation of Rosenthal fibers relative to a subject that does not have AxD. In some embodiments, the subject has increased GFAP expression in the brain relative to a subject that does not have AxD. In some embodiments, the subject has increased GFAP expression in the hippocampus and/or olfactory bulbs relative to a subject that does not have AxD. In some embodiments, the subject has increased Rosenthal fiber expression in the brain relative to a subject that does not have AxD. In some embodiments, the subject has increased Rosenthal fibers formation in the hippocampus and/or olfactory bulbs relative to a subject that does not have AxD.

In some embodiments, methods provided herein results in decreased GFAP expression and/or activity (e.g., by at least 10%, at least 20%, at least 30%, at least 40%, at least 50%, at least 60%, at least 70%, at least 80%, at least 90%, at least 95%, or 100%) in the brain (e.g., hippocampus and/or olfactory bulbs) in a subject having AxD relative to the subject prior to the administration. Expression and/or activity of GFAP can be measured by suitable known methods in the art, e.g., an immunoassay (e.g., Enzyme-linked immunosorbent assay (ELISA) or Chemiluminescent immunoassay (CLIA)) or real-time polymerase chain reaction (RT-PCR).

In some embodiments, methods provided herein results in decreased Rosenthal fiber formation (e.g., by at least 10%, at least 20%, at least 30%, at least 40%, at least 50%, at least 60%, at least 70%, at least 80%, at least 90%, at least 95%, or 100%) in the brain (e.g., hippocampus and/or olfactory bulbs) in a subject having AxD relative to the subject prior to the administration. Formation of Rosenthal fibers can be measured by suitable known methods in the art, e.g., Magnetic Resonance Imaging (MRI), immunostaining of a brain biopsy (e.g., immunohistochemistry, immunofluorescence or western blot).

### Modes of Administration

The rAAVs of the disclosure may be delivered to a subject in compositions according to any appropriate methods known in the art. For example, an rAAV, preferably suspended in a physiologically compatible carrier (i.e., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, rAAV as described in the disclosure are administered by intravenous injection. In some embodiments, the rAAV are administered by intracerebral injection. In some embodiments, the rAAV are administered by intrathecal injection. In some embodiments, the rAAV are administered by intrastriatal injection. In some embodiments, the rAAV are delivered by intracranial injection. In some embodiments, the rAAV are delivered by cisterna magna injection. In some embodiments, the rAAV are delivered by cerebral lateral ventricle injection.

Aspects of the instant disclosure relate to compositions comprising a recombinant AAV comprising a capsid protein and a nucleic acid encoding a transgene, wherein the transgene comprises a nucleic acid sequence encoding one or more miRNAs. In some embodiments, each miRNA comprises a sequence set forth in any one of SEQ ID NOs: 1 or 2. In some embodiments, the nucleic acid further comprises AAV ITRs. In some embodiments, the rAAV comprises an rAAV vector represented by the sequence set forth in any one of SEQ ID NO: 4 or 5, or a portion thereof. In some embodiments, a composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, intrathecal, intracranial, intracerebroventricular, and other parental routes of administration. Routes of administration may be combined, if desired.

The dose of rAAV virions required to achieve a particular “therapeutic effect.” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In certain embodiments, 10¹² or 10¹³ rAAV genome copies is effective to target CNS tissue. In some cases, stable transgenic animals are produced by multiple doses of an rAAV.

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and, include, for example, addition of surfactants, pH adjustment, salt concentration adjustment, etc. (See, e.g., Wright FR, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, intracranially, intracerebroventricularly, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, the instant disclosure relates to a kit for producing a rAAV, the kit comprising a container housing an isolated nucleic acid comprising an miRNA comprising or encoded by the sequence set forth in any one of SEQ ID NOs: 1 or 2. In some embodiments, the kit further comprises a container housing an isolated nucleic acid encoding an AAV capsid protein, for example an AAV9 capsid protein.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

Exemplary embodiments of the invention will be described in more detail by the following examples. These embodiments are exemplary of the invention, which one skilled in the art will recognize is not limited to the exemplary embodiments.

### EXAMPLE

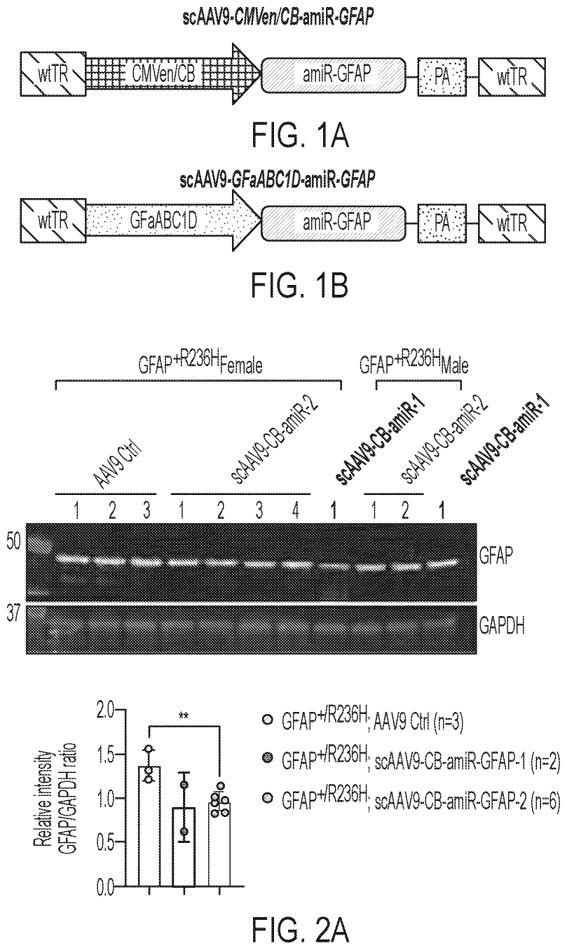

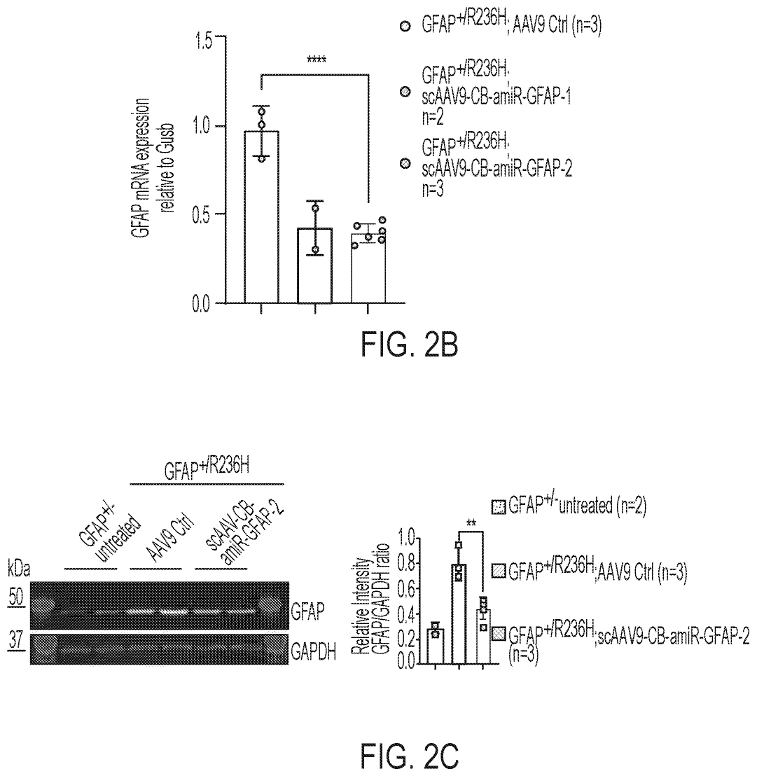

This example describes artificial mRNAs (amiRNAs or amiR) designed to target selected GFAP sequences in mouse, rat, and human. Multiple amiR-GFAP constructs that can efficiently silence the expression of GFAP in all three species were tested in vitro. Self-complementary rAAV vectors encoding certain amiR-GFAP designs (referred to as #1 and #2), under the control of the ubiquitous chicken beta actin promoter with CMV enhancer (CMVen/CB) were packaged into AAV serotype 9 (AAV9) (FIG. 1A) and injected five-week-old Alexander Disease (AxD) disease mice (GFAP^{+/R236H}) by intravenous administration at a dose of 2×10¹⁴ genome copies (GCs)/kg. Three weeks later, it was observed that both scAAV9-CMVen/CB-amiR-GFAP vectors down regulated GFAP mRNA and protein in the brain 50% more than the control vector (FIGS. 2A-2C). In addition, histopathologic analysis of treated mouse brain showed reduction of Rosenthal fibers.

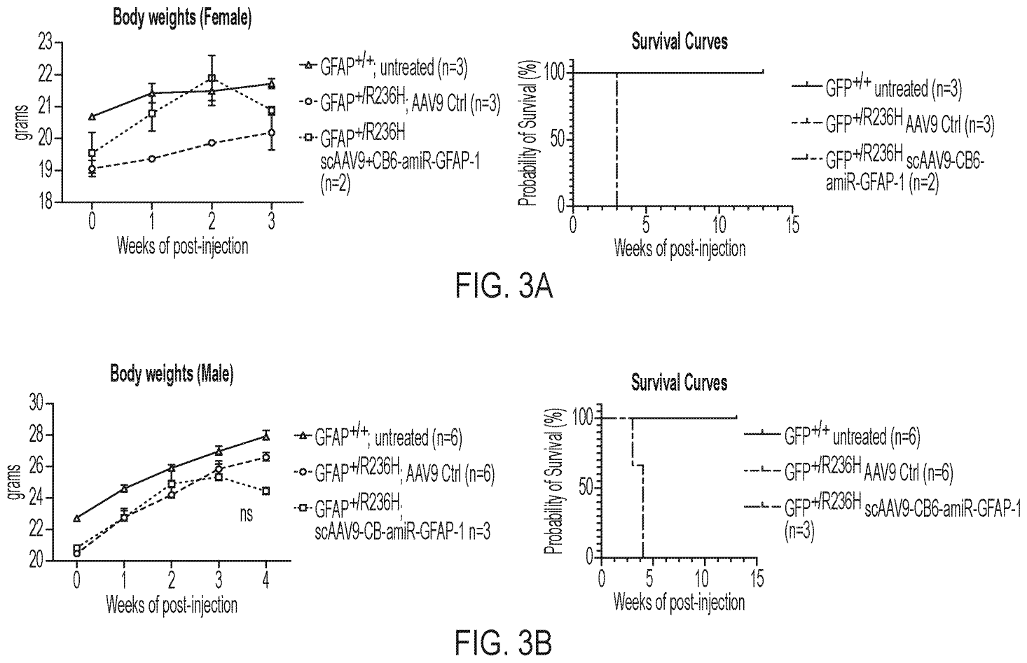

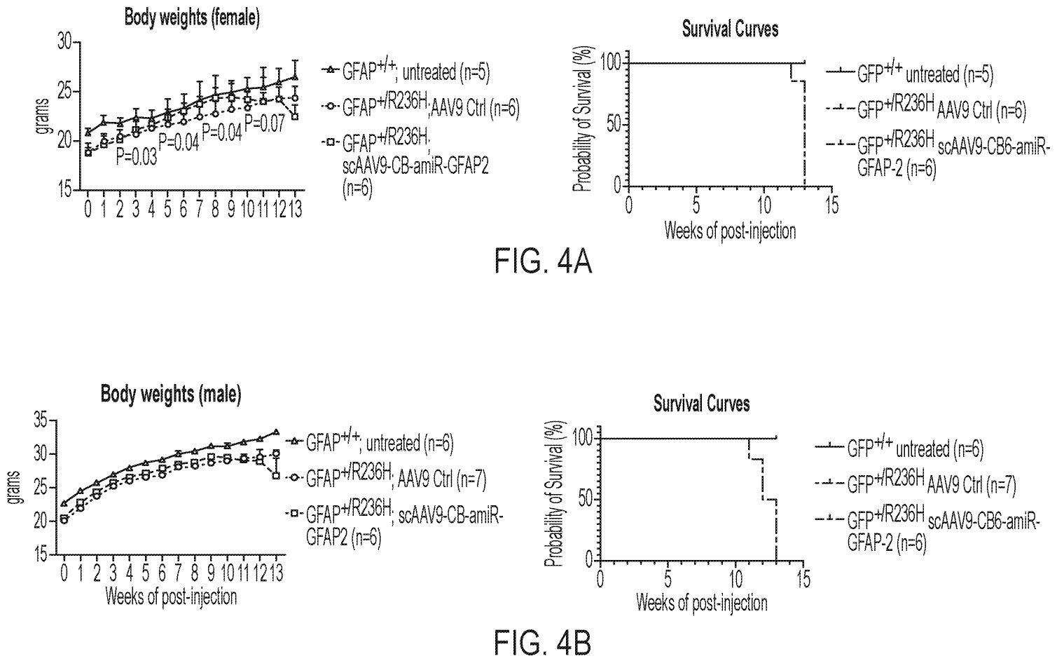

To monitor long-term therapeutic efficacy and safety, the treatment was extended to three months. It was observed that scAAV9-CMVen/CB-amiR-GFAP #1-treated mice started dying from digestive disorders around three to four weeks post-treatment (FIGS. 3A-3B). Interestingly, GFAP^{+/R236H} mice treated with scAAV9-CMVen/CB-amiR-GFAP #2 showed improved body weights until the tenth week post-injection (FIGS. 4A-4B). During this period, the animals started losing weight, hunching, and abnormally rapid breathing. Necropsy of these animals revealed enlarged hearts, as well as reduced GFAP expression in the brain as compared to control vector-treated mice.

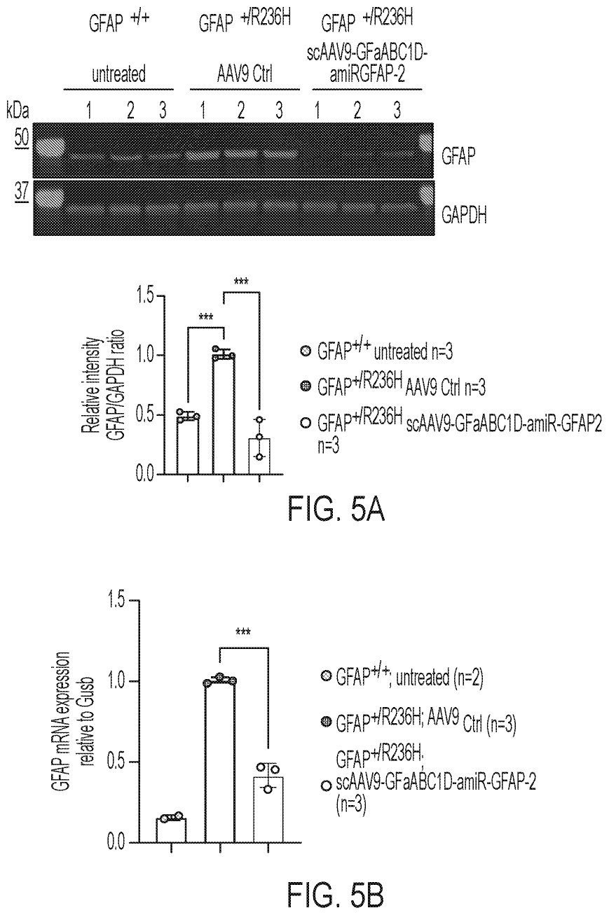

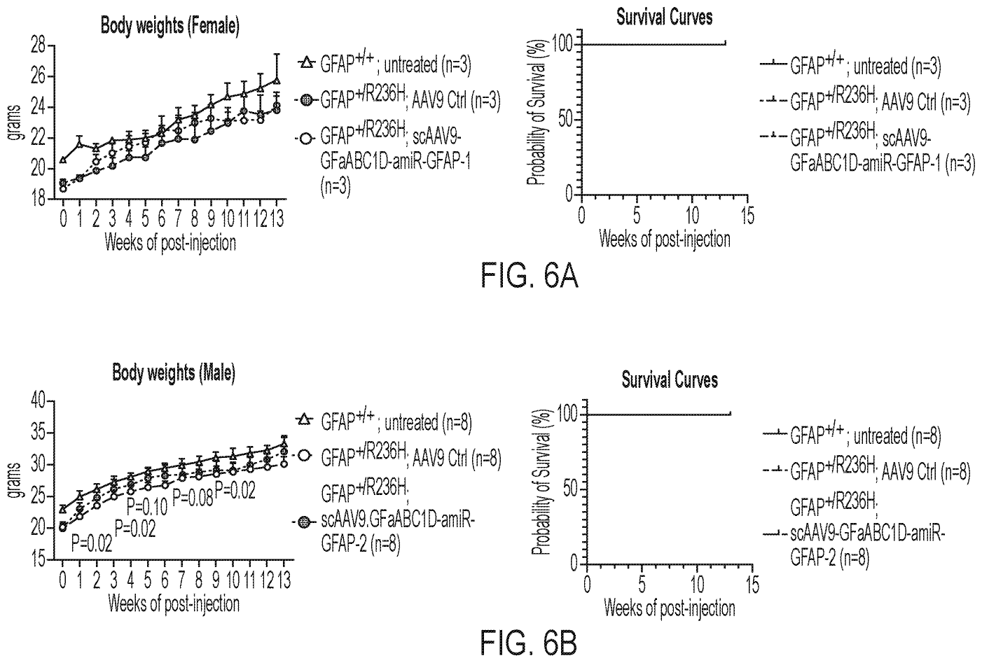

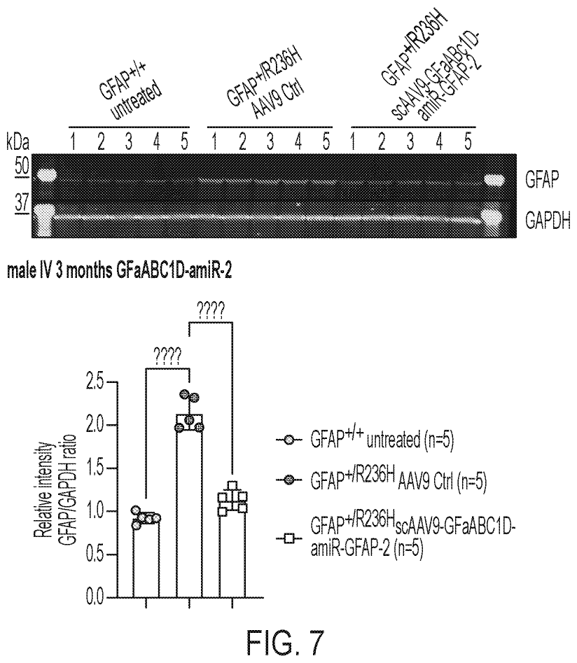

It was observed that expression of amiR-GFAP in non-astrocyte cells might have resulted in the aforementioned side effects. To reduce off-target silencing, the CMVen/CB promoter was replaced with an endogenous GFAP promoter (e.g., GFaABC1D) in the amiR-GFAP vector to drive astrocyte-specific expression. The second generation of amiR-GFAP #1 vector not only eliminated the early death, but also improved weight gains during development as early as three weeks after injection. Treatments with the second generation of amiR-GFAP #2 vector also improved the body weights of GFAP^{+/R236H} mice (FIGS. 6A-6B). Western blot and qRT-PCR analysis revealed that GFAP protein and mRNA levels were reduced to 30% at three weeks post-injection in the amiR-GFAP #2 vector treated mice (FIGS. 5A-5B). The long-term monitoring of animals that received the second-generation vectors was continued for three months. Western blot analysis revealed that GFAP protein levels were reduced 50% compared at three months post-injection in the amiR-GFAP #2 vector treated mice, bringing back the GFAP protein level comparable to the healthy litter mates (FIG. 7).

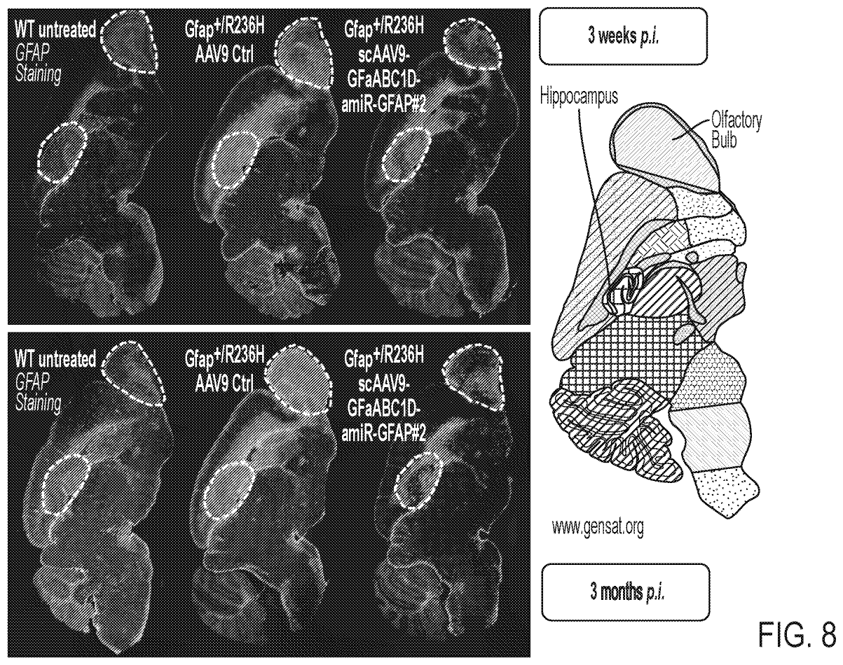

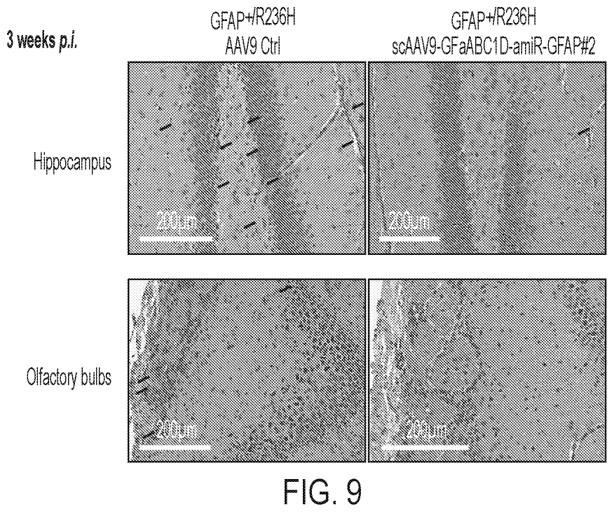

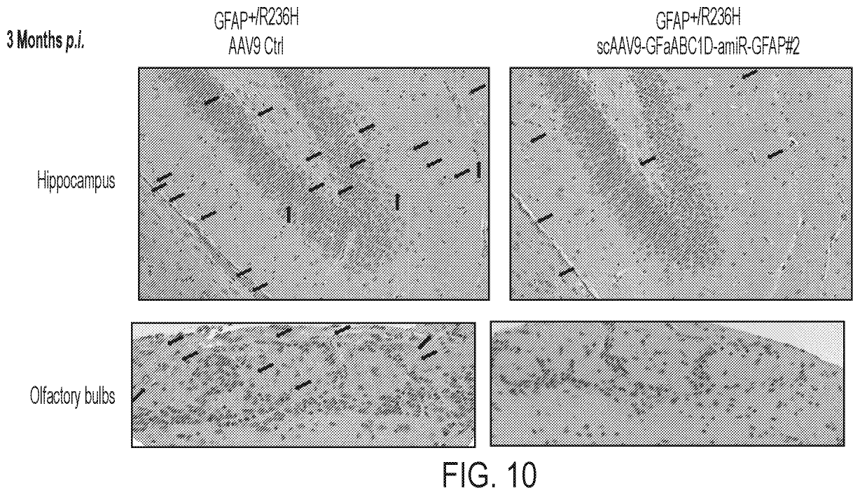

Next, to look at which specific brain regions were affected GFAP immunofluorescence staining was performed at three weeks and three months post injection. Treatment with the second generation of amiR-GFAP #2 vector reduced GFAP expression in the hippocampus and olfactory bulbs, both critical for AxD pathology, at three weeks and three months post injection (FIG. 8). In addition, histopathologic analysis of the brains of second generation of amiR-GFAP #2 treated mice showed a reduction of Rosenthal fibers in hippocampus and olfactory bulbs after 3 weeks and 3 months treatment (FIGS. 9 and 10). Together these results indicate that treatment with amiR-GFAP #2 reduces GFAP expression and Rosenthal fiber expression in both the hippocampus and olfactory bulbs, which are hallmarks of AxD.

### REPRESENTATIVE SEQUENCES

>amiR-GFAP-2 (Guide Strand is bold; Passenger Strand is underlined)
(SEQ ID NO: 1)
AGGGCTCTGCGTTTGCTCCAGGTAGTCCGCTGCTCCCTTGGGCCTGGGCCCACTGACAGCCCTG
GTGCCTCTGGCCGGCTGCACACCTCCTGGCGGGCAGCTGTGTCATTGAGCTCCATCATCTCTTG
TTCTGGCAATACCTGAGAGATGAACGGGCTCAATGACACGGAGGCCTGCCCTGACTGCCCACGG
TGCCGTGGCCAAAGAGGATCTAAGGGCACCGCTGAGGGCCTACCTAACCATCGTGGGGAATAAG
GACAGTGTCACCC
>amiR-GFAP-1 (Guide Strand is bold; Passenger Strand is underlined)
(SEQ ID NO: 2)
AGGGCTCTGCGTTTGCTCCAGGTAGTCCGCTGCTCCCTTGGGCCTGGGCCCACTGACAGCCCTG
GTGCCTCTGGCCGGCTGCACACCTCCTGGCGGGCAGCTGTGTTGTTTTGCTGTTCCAGGAAGTG
TTCTGGCAATACCTGCTTCCTGGTTCGGCAAAACAACACGGAGGCCTGCCCTGACTGCCCACGG
TGCCGTGGCCAAAGAGGATCTAAGGGCACCGCTGAGGGCCTACCTAACCATCGTGGGGAATAAG
GACAGTGTCACCC
>GfaABC1D promoter
(SEQ ID NO: 3)
AACATATCCTGGTGTGGAGTAGGGGACGCTGCTCTGACAGAGGCTCGGGGGCCTGAGCTGGCTC
TGTGAGCTGGGGAGGAGGCAGACAGCCAGGCCTTGTCTGCAAGCAGACCTGGCAGCATTGGGCT
GGCCGCCCCCCAGGGCCTCCTCTTCATGCCCAGTGAATGACTCACCTTGGCACAGACACAATGT
TCGGGGTGGGCACAGTGCCTGCTTCCCGCCGCACCCCAGCCCCCCTCAAATGCCTTCCGAGAAG
CCCATTGAGCAGGGGGCTTGCATTGCACCCCAGCCTGACAGCCTGGCATCTTGGGATAAAAGCA
GCACAGCCCCCTAGGGGCTGCCCTTGCTGTGTGGCGCCACCGGCGGTGGAGAACAAGGCTCTAT
TCAGCCTGTGCCCAGGAAAGGGGATCAGGGGATGCCCAGGCATGGACAGTGGGTGGCAGGGGGG
GAGAGGAGGGCTGTCTGCTTCCCAGAAGTCCAAGGACACAAATGGGTGAGGGGAGAGCTCTCCC
CATAGCTGGGCTGCGGCCCAACCCCACCCCCTCAGGCTATGCCAGGGGGTGTTGCCAGGGGCAC
CCGGGCATCGCCAGTCTAGCCCACTCCTTCATAAAGCCCTCGCATCCCAGGAGCGAGCAGAGCC
AGAGCAGGTTGGAGAGGAGACGCATCACCTCCGCTGCTCGC
>rAAV_amiR-GFAP-2
(SEQ ID NO: 4)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGTAGCCATGCTCTAGGAAGATCAA
TTCGGTACAATTCACGCGTCGACATTGATTATTGACTCTGGTCAACATATCCTGGTGTGGAGTA
GGGGACGCTGCTCTGACAGAGGCTCGGGGGCCTGAGCTGGCTCTGTGAGCTGGGGAGGAGGCAG
ACAGCCAGGCCTTGTCTGCAAGCAGACCTGGCAGCATTGGGCTGGCCGCCCCCCAGGGCCTCCT
CTTCATGCCCAGTGAATGACTCACCTTGGCACAGACACAATGTTCGGGGTGGGCACAGTGCCTG
CTTCCCGCCGCACCCCAGCCCCCCTCAAATGCCTTCCGAGAAGCCCATTGAGCAGGGGGCTTGC
ATTGCACCCCAGCCTGACAGCCTGGCATCTTGGGATAAAAGCAGCACAGCCCCCTAGGGGCTGC
CCTTGCTGTGTGGCGCCACCGGCGGTGGAGAACAAGGCTCTATTCAGCCTGTGCCCAGGAAAGG
GGATCAGGGGATGCCCAGGCATGGACAGTGGGTGGCAGGGGGGGAGAGGAGGGCTGTCTGCTTC
CCAGAAGTCCAAGGACACAAATGGGTGAGGGGAGAGCTCTCCCCATAGCTGGGCTGCGGCCCAA
CCCCACCCCCTCAGGCTATGCCAGGGGGTGTTGCCAGGGGCACCCGGGCATCGCCAGTCTAGCC
CACTCCTTCATAAAGCCCTCGCATCCCAGGAGCGAGCAGAGCCAGAGCAGGTTGGAGAGGAGAC
GCATCACCTCCGCTGCTCGCCACCGCGGTGGCGGCCCTAGAGTCGATCGAGGAACTGAAAAACC
AGAAAGTTAACTGGTAAGTTTAGTCTTTTTGTCTTTTATTTCAGGTCCCAGGGCTCTGCGTTTG
CTCCAGGTAGTCCGCTGCTCCCTTGGGCCTGGGCCCACTGACAGCCCTGGTGCCTCTGGCCGGC
TGCACACCTCCTGGCGGGCAGCTGTGTCATTGAGCTCCATCATCTCTTGTTCTGGCAATACCTG
AGAGATGAACGGGCTCAATGACACGGAGGCCTGCCCTGACTGCCCACGGTGCCGTGGCCAAAGA
GGATCTAAGGGCACCGCTGAGGGCCTACCTAACCATCGTGGGGAATAAGGACAGTGTCACCCGG
ATCCGGTGGTGGTGCAAATCAAAGAACTGCTCCTCAGTGGATGTTGCCTTTACTTCTAGGCCTG
TACGGAAGTGTTACTTCTGCTCTAAAAGCTGCGGAATTGTACCCGCGGCCGATCCACCGGAGCT
TATCGATACCGTCGACTAGAGCTCGCTGATCAGCCTCGACTGTGCCTTCTAGTTGCCAGCCATC
TGTTGTTTGCCCCTCCCCCGTGCCTTCCTTGACCCTGGAAGGTGCCACTCCCACTGTCCTTTCC
TAATAAAATGAGGAAATTGCATCGCATTGTCTGAGTAGGTGTCATTCTATTCTGGGGGGTGGGG
TGGGGCAGGACAGCAAGGGGGAGGATTGGGAAGACAATTAGGTAGATAAGTAGCATGGCGGGTT
AATCATTAACTACAAGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCGGGCGGCCTCAGTGA
GCGAGCGAGCGCGCAG
>rAAV_amiR-GFAP-1
(SEQ ID NO: 5)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGTAGCCATGCTCTAGGAAGATCAA
TTCGGTACAATTCACGCGTCGACATTGATTATTGACTCTGGTCAACATATCCTGGTGTGGAGTA
GGGGACGCTGCTCTGACAGAGGCTCGGGGGCCTGAGCTGGCTCTGTGAGCTGGGGAGGAGGCAG
ACAGCCAGGCCTTGTCTGCAAGCAGACCTGGCAGCATTGGGCTGGCCGCCCCCCAGGGCCTCCT
CTTCATGCCCAGTGAATGACTCACCTTGGCACAGACACAATGTTCGGGGTGGGCACAGTGCCTG
CTTCCCGCCGCACCCCAGCCCCCCTCAAATGCCTTCCGAGAAGCCCATTGAGCAGGGGGCTTGC
ATTGCACCCCAGCCTGACAGCCTGGCATCTTGGGATAAAAGCAGCACAGCCCCCTAGGGGCTGC
CCTTGCTGTGTGGCGCCACCGGCGGTGGAGAACAAGGCTCTATTCAGCCTGTGCCCAGGAAAGG
GGATCAGGGGATGCCCAGGCATGGACAGTGGGTGGCAGGGGGGGAGAGGAGGGCTGTCTGCTTC
CCAGAAGTCCAAGGACACAAATGGGTGAGGGGAGAGCTCTCCCCATAGCTGGGCTGCGGCCCAA
CCCCACCCCCTCAGGCTATGCCAGGGGGTGTTGCCAGGGGCACCCGGGCATCGCCAGTCTAGCC
CACTCCTTCATAAAGCCCTCGCATCCCAGGAGCGAGCAGAGCCAGAGCAGGTTGGAGAGGAGAC
GCATCACCTCCGCTGCTCGCCACCGCGGTGGCGGCCCTAGAGTCGATCGAGGAACTGAAAAACC
AGAAAGTTAACTGGTAAGTTTAGTCTTTTTGTCTTTTATTTCAGGTCCCAGGGCTCTGCGTTTG
CTCCAGGTAGTCCGCTGCTCCCTTGGGCCTGGGCCCACTGACAGCCCTGGTGCCTCTGGCCGGC
TGCACACCTCCTGGCGGGCAGCTGTGTTGTTTTGCTGTTCCAGGAAGTGTTCTGGCAATACCTG
CTTCCTGGTTCGGCAAAACAACACGGAGGCCTGCCCTGACTGCCCACGGTGCCGTGGCCAAAGA
GGATCTAAGGGCACCGCTGAGGGCCTACCTAACCATCGTGGGGAATAAGGACAGTGTCACCCGG
ATCCGGTGGTGGTGCAAATCAAAGAACTGCTCCTCAGTGGATGTTGCCTTTACTTCTAGGCCTG
TACGGAAGTGTTACTTCTGCTCTAAAAGCTGCGGAATTGTACCCGCGGCCGATCCACCGGAGCT
TATCGATACCGTCGACTAGAGCTCGCTGATCAGCCTCGACTGTGCCTTCTAGTTGCCAGCCATC
TGTTGTTTGCCCCTCCCCCGTGCCTTCCTTGACCCTGGAAGGTGCCACTCCCACTGTCCTTTCC
TAATAAAATGAGGAAATTGCATCGCATTGTCTGAGTAGGTGTCATTCTATTCTGGGGGGTGGGG
TGGGGCAGGACAGCAAGGGGGAGGATTGGGAAGACAATTAGGTAGATAAGTAGCATGGCGGGTT
AATCATTAACTACAAGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCGGGCGGCCTCAGTGA
GCGAGCGAGCGCGCAG
Human GFAP NM_002055.5 (target region for amiR-GFAP-1 underlined;
target region for amiR-GFAP-2 in bold)
(SEQ ID NO: 6)
AGAGCCAGAGCAGGATGGAGAGGAGACGCATCACCTCCGCTGCTCGCCGCTCCTACGTCTCCTC
AGGGGAGATGATGGTGGGGGGCCTGGCTCCTGGCCGCCGTCTGGGTCCTGGCACCCGCCTCTCC
CTGGCTCGAATGCCCCCTCCACTCCCGACCCGGGTGGATTTCTCCCTGGCTGGGGCACTCAATG
CTGGCTTCAAGGAGACCCGGGCCAGTGAGCGGGCAGAGATGATGGAGCTCAATGACCGCTTTGC
CAGCTACATCGAGAAGGTTCGCTTCCTGGAACAGCAAAACAAGGCGCTGGCTGCTGAGCTGAAC
CAGCTGCGGGCCAAGGAGCCCACCAAGCTGGCAGACGTCTACCAGGCTGAGCTGCGAGAGCTGC
GGCTGCGGCTCGATCAACTCACCGCCAACAGCGCCCGGCTGGAGGTTGAGAGGGACAATCTGGC
ACAGGACCTGGCCACTGTGAGGCAGAAGCTCCAGGATGAAACCAACCTGAGGCTGGAAGCCGAG
AACAACCTGGCTGCCTATAGACAGGAAGCAGATGAAGCCACCCTGGCCCGTCTGGATCTGGAGA
GGAAGATTGAGTCGCTGGAGGAGGAGATCCGGTTCTTGAGGAAGATCCACGAGGAGGAGGTTCG
GGAACTCCAGGAGCAGCTGGCCCGACAGCAGGTCCATGTGGAGCTTGACGTGGCCAAGCCAGAC
CTCACCGCAGCCCTGAAAGAGATCCGCACGCAGTATGAGGCAATGGCGTCCAGCAACATGCATG
AAGCCGAAGAGTGGTACCGCTCCAAGTTTGCAGACCTGACAGACGCTGCTGCCCGCAACGCGGA
GCTGCTCCGCCAGGCCAAGCACGAAGCCAACGACTACCGGCGCCAGTTGCAGTCCTTGACCTGC
GACCTGGAGTCTCTGCGCGGCACGAACGAGTCCCTGGAGAGGCAGATGCGCGAGCAGGAGGAGC
GGCACGTGCGGGAGGCGGCCAGTTATCAGGAGGCGCTGGCGCGGCTGGAGGAAGAGGGGCAGAG
CCTCAAGGACGAGATGGCCCGCCACTTGCAGGAGTACCAGGACCTGCTCAATGTCAAGCTGGCC
CTGGACATCGAGATCGCCACCTACAGGAAGCTGCTAGAGGGCGAGGAGAACCGGATCACCATTC
CCGTGCAGACCTTCTCCAACCTGCAGATTCGAGAAACCAGCCTGGACACCAAGTCTGTGTCAGA
AGGCCACCTCAAGAGGAACATCGTGGTGAAGACCGTGGAGATGCGGGATGGAGAGGTCATTAAG
GAGTCCAAGCAGGAGCACAAGGATGTGATGTGAGGCAGGACCCACCTGGTGGCCTCTGCCCCGT
CTCATGAGGGGCCCGAGCAGAAGCAGGATAGTTGCTCCGCCTCTGCTGGCACATTTCCCCAGAC
CTGAGCTCCCCACCACCCCAGCTGCTCCCCTCCCTCCTCTGTCCCTAGGTCAGCTTGCTGCCCT
AGGCTCCGTCAGTATCAGGCCTGCCAGACGGCACCCACCCAGCACCCAGCAACTCCAACTAACA
AGAAACTCACCCCCAAGGGGCAGTCTGGAGGGGCATGGCCAGCAGCTTGCGTTAGAATGAGGAG
GAAGGAGAGAAGGGGAGGAGGGCGGGGGGCACCTACTACATCGCCCTCCACATCCCTGATTCCT
GTTGTTATGGAAACTGTTGCCAGAGATGGAGGTTCTCTCGGAGTATCTGGGAACTGTGCCTTTG
AGTTTCCTCAGGCTGCTGGAGGAAAACTGAGACTCAGACAGGAAAGGGAAGGCCCCACAGACAA
GGTAGCCCTGGCCAGAGGCTTGTTTTGTCTTTTGGTTTTTATGAGGTGGGATATCCCTATGCTG
CCTAGGCTGACCTTGAACTCCTGGGCTCAAGCAGTCTACCCACCTCAGCCTCCTGTGTAGCTGG
GATTATAGATTGGAGCCACCATGCCCAGCTCAGAGGGTTGTTCTCCTAGACTGACCCTGATCAG
TCTAAGATGGGTGGGGACGTCCTGCCACCTGGGGCAGTCACCTGCCCAGATCCCAGAAGGACCT
CCTGAGCGATGACTCAAGTGTCTCAGTCCACCTGAGCTGCCATCCAGGGATGCCATCTGTGGGC
ACGCTGTGGGCAGGTGGGAGCTTGATTCTCAGCACTTGGGGGATCTGTTGTGTACGTGGAGAGG
GATGAGGTGCTGGGAGGGATAGAGGGGGGCTGCCTGGCCCCCAGCTGTGGGTACAGAGAGGTCA
AGCCCAGGAGGACTGCCCCGTGCAGACTGGAGGGGACGCTGGTAGAGATGGAGGAGGAGGCAAT
TGGGATGGCGCTAGGCATACAAGTAGGGGTTGTGGGTGACCAGTTGCACTTGGCCTCTGGATTG
TGGGAATTAAGGAAGTGACTCATCCTCTTGAAGATGCTGAAACAGGAGAGAAAGGGGATGTATC
CATGGGGGCAGGGCATGACTTTGTCCCATTTCTAAAGGCCTCTTCCTTGCTGTGTCATACCAGG
CCGCCCCAGCCTCTGAGCCCCTGGGACTGCTGCTTCTTAACCCCAGTAAGCCACTGCCACACGT
CTGACCCTCTCCACCCCATAGTGACCGGCTGCTTTTCCCTAAGCCAAGGGCCTCTTGCGGTCCC
TTCTTACTCACACACAAAATGTACCCAGTATTCTAGGTAGTGCCCTATTTTACAATTGTAAAAC
TGAGGCACGAGCAAAGTGAAGACACTGGCTCATATTCCTGCAGCCTGGAGGCCGGGTGCTCAGG
GCTGACACGTCCACCCCAGTGCACCCACTCTGCTTTGACTGAGCAGACTGGTGAGCAGACTGGT
GGGATCTGTGCCCAGAGATGGGACTGGGAGGGCCCACTTCAGGGTTCTCCTCTCCCCTCTAAGG
CCGAAGAAGGGTCCTTCCCTCTCCCCAAGACTTGGTGTCCTTTCCCTCCACTCCTTCCTGCCAC
CTGCTGCTGCTGCTGCTGCTAATCTTCAGGGCACTGCTGCTGCCTTTAGTCGCTGAGGAAAAAT
AAAGACAAATGCTGCGCCCTTCCCCAGAGTGGACTCTGATCTGTTCATGAGAGGGCGGGACTGG
GGCCAAGATGTAGCCTTTGACAAGACCAACTCATTTCTTATTACTGATCATCTCTGGGGCCCAT
GCCCTCACCAAATTCCACCCGCAGCCAAAGAGGACATACACCAGCTCCCTCCACTCTTTTCTTC
CTTCCTCTCCCTGCTACCTGCAACTCAACCAGCACAATCTTCATAGGCAAGAAAGCAAAGCAGC
TCAAACATGATTCAACACTGATCAGTGTTTACCACTGGATAAATCTGAGTTCACACTTTCCTTC
TCTGACCTAAATGTGAAGTCAGGAAACACATGTGCCCTACTTCCATCCTGAGCTCAGTCCCCAA
TCTCCCACCAGCCTCAGGCCCCTCCACTTCTCAGATCAGGTCCCAGACCTGCCCATGAAAATGG
GGAGCAGGCTGTAACAGATTTGTCCACATGTTCCTACCACCTGTCCCAACCCAGGGTACCCACC
CAGAGACATCTGGTATCATTTAACAAACACATTGAAGGACAACTGGTCTTCAGAGCTGAAGAGA
GCTCCTAGGGGGAGAAGCTGGGACAACAGTGAAATAAGTAGCAGCAGCAACGACAGAAGTGAAT
GGTGACAAAGACTGCTGTGATGAGCAGGTAGCCTATCAGGGTGAGCTCCACAGCCGAGCGAGTC
TCAGGATCTGAGAACGAGGCTGGGTAGTGCCCATGAGATGTCACACCCAGCCGGAAGCCAGCAA
CTAGCACACCCTGCCTCCAGCAATAGTAGATGCCCCGGTCATCCAGCTGGGTGAAGCGGATGTG
GAGCTGGTTGCCGTGGTCAATGAACACCCTCATGGACCTGTTGACACCCTTCAGGTACTGTGTG
CGGTAGAGGTGCTGGCGGTCTTTGTCCCAGGCCACTGCATGCTCTGGCCGGGCCCCAGGACAGG
AGATGATGAGTCCATGGCCCAGTCTCTGCTGGTGGAACTGAATGGGCACCTGGGGCACCCAGGG
CCGGCTGCCCACTTTGGACACATAGTTAATGATGGCCAGCACGCCCTCCCGGATGGTCTTTGTC
TTCTCACAGGGTACTAAGCAGCTCCGAACCAGCACCTCAGGCGTGTGGTCCCTGGCCTTGGTCC
GCAGCTTCCTTGGCACAGCCCTTGAGCCACAAGACACCACATCGGGCACGGCCTTGAGGTAGCG
TGGGGAGAGGTCTGGGCTCTGCAGGTAGCAGAGGCCGATGCGCCACTGCTCCCCACGCACTCCG
CAGCGGTCACAGGGGGTCCATTCCCAGAAGGTGGTGAAGACATGGAGGTGCCCATAGTATTCAT
CTGCAAAGGGCTCCTGGCCCTTGTCCTGGAAAGTGGCCACCATTCCCTCACTGTTCTGGATGTC
CACATCGTAGGCGTAAAAGTAGTCCCCCTTGCGGGTGCCGCAGAAGTACAGGCCTGAGTCCTCA
GACTGAGCCCTGAAAACCAACAAGCTGAACATGCGGATGCTGAAGCGGGTCAGCATGTCGCTGC
CCACACGTACCTGGGCTGCCTCCGTCAGCACCCGCCCATCAAAGTCCGTCAGCACTTTGGTGTG
GCTGCTACCTAGGTGCTTTTGGTAGAACCAGACTACAGCTGGCACCTCTTCGGGTTTGCAGTGA
CAGGGAAGCTCAAAGCTCATGTCGGCCAGGTAGGCTGCATTTTCAAACATCAGGAAAGCAGGGC
AGGGGGTCCTCTGAAAAATGTTTTCCTTCTCCACAATTTCAAAGGCCTGGAGCCCCCATGCCCA
CAGGAGCACAGTGGTGAGGGCCAGGTGCATACCTGAAGGAGGCAGGGGTCAGAGGGGCAGGGCA
AAACCAGGGCATTAAAGGCTCATAGGGCTCCTAGAAAGCTCTGCTAAGCGGAAGCCTCTAGATG
AGGAAAGGATTATGCAGCCAGGAAAAGCAGCAACAATCTGCAGAGGAAGCCGCCAAGTGCAAGG
CAATTTATTCCCAGTGGATGTACAAGATGCCCTTCTAACATTCCAGACCTGATCTCAGGGTGGG
GGGGGAAAGCCATTCTAGAACCTGGCCTTTACTCCCCTTTCTAGAACACTGGCGCTCACCCAAG
AATGGGTCAAAGGAAACCGGAATGAGAAGGGCGGGCCGAGGTGCTCGGGCAGGGAGATCTCTGC
CTCAGTGCTCCAGGCCCTGCCCTGCCAGCCTGGTGGAAAAGTCTTTCATCAACCTGGGGGATGA
AGGAAACCCACCCTCCTGCATATCTGGCCATCCGGGAGGCTGGCTGGACCTGAGCTGATGGCTT
GGGACTTTCCCAGGCCCAACCTGCACAAGAACTGAGTCTCTAGGGGAAAATTCAACACCTCAAA
TGATGTAGTATTTGATCATTTGTTGATTACATGTCCATTCATTGGTTTGGGGCTATAAACATTC
TTGTTAAGAGCTGTGGAGATCAGTGTTTGTTTACCATAAAGATTTTGCTTTTTCCCTTTTA
Human GFAP NM_001131019.3 (target region for amiR-GFAP-1 underlined;
target region for amiR-GFAP-2 in bold)
(SEQ ID NO: 7)
AGAGCCAGAGCAGGATGGAGAGGAGACGCATCACCTCCGCTGCTCGCCGCTCCTACGTCTCCTC
AGGGGAGATGATGGTGGGGGGCCTGGCTCCTGGCCGCCGTCTGGGTCCTGGCACCCGCCTCTCC
CTGGCTCGAATGCCCCCTCCACTCCCGACCCGGGTGGATTTCTCCCTGGCTGGGGCACTCAATG
CTGGCTTCAAGGAGACCCGGGCCAGTGAGCGGGCAGAGATGATGGAGCTCAATGACCGCTTTGC
CAGCTACATCGAGAAGGTTCGCTTCCTGGAACAGCAAAACAAGGCGCTGGCTGCTGAGCTGAAC
CAGCTGCGGGCCAAGGAGCCCACCAAGCTGGCAGACGTCTACCAGGCTGAGCTGCGAGAGCTGC
GGCTGCGGCTCGATCAACTCACCGCCAACAGCGCCCGGCTGGAGGTTGAGAGGGACAATCTGGC
ACAGGACCTGGCCACTGTGAGGCAGAAGCTCCAGGATGAAACCAACCTGAGGCTGGAAGCCGAG
AACAACCTGGCTGCCTATAGACAGGAAGCAGATGAAGCCACCCTGGCCCGTCTGGATCTGGAGA
GGAAGATTGAGTCGCTGGAGGAGGAGATCCGGTTCTTGAGGAAGATCCACGAGGAGGAGGTTCG
GGAACTCCAGGAGCAGCTGGCCCGACAGCAGGTCCATGTGGAGCTTGACGTGGCCAAGCCAGAC
CTCACCGCAGCCCTGAAAGAGATCCGCACGCAGTATGAGGCAATGGCGTCCAGCAACATGCATG
AAGCCGAAGAGTGGTACCGCTCCAAGTTTGCAGACCTGACAGACGCTGCTGCCCGCAACGCGGA
GCTGCTCCGCCAGGCCAAGCACGAAGCCAACGACTACCGGCGCCAGTTGCAGTCCTTGACCTGC
GACCTGGAGTCTCTGCGCGGCACGAACGAGTCCCTGGAGAGGCAGATGCGCGAGCAGGAGGAGC
GGCACGTGCGGGAGGCGGCCAGTTATCAGGAGGCGCTGGCGCGGCTGGAGGAAGAGGGGCAGAG
CCTCAAGGACGAGATGGCCCGCCACTTGCAGGAGTACCAGGACCTGCTCAATGTCAAGCTGGCC
CTGGACATCGAGATCGCCACCTACAGGAAGCTGCTAGAGGGCGAGGAGAACCGGATCACCATTC
CCGTGCAGACCTTCTCCAACCTGCAGATTCGAGGGGGCAAAAGCACCAAAGACGGGGAAAATCA
CAAGGTCACAAGATATCTCAAAAGCCTCACAATACGAGTTATACCAATACAGGCTCACCAGATT
GTAAATGGAACGCCGCCGGCTCGCGGTTAGCTGCCTGCCTCTCAGACACGGCGCTTTGCCCAGC
TTGACAGGGAGTGAGCCTCACCCACCCCATCCTCCCAATCCCCCTGAGTTCCCTCTTCCCAGGC
TTCCCCTAAAGGGCCTGGACTGCGTCATTTTCCCAGGAACTGCAGTGCCCAGCCCAGGACGTGG
TACAGAGTAACTGTACATTAAACTGGCAGAGCTTGTTAGTGGTAAAGGTGGTGAGTCCTTGGGT
GCGCAGTGGAGCTGCTCTGGGGCCTCTGAGCAAGCAGCAGCCTCTGTCTCACCTCTTCCTGTCA
CTGGGAGGGCCCCTTGGGTCTCGCTGTGCCTGGACGCCAGGCTCTCTGCTTTATTCTTTCATCC
CTGAGGCTCCATCGCTCAGCTCAGTGCTGACTCAGTTCAGAGGATTCTTCCCTCAGGACCGCAG
CTCTTGCAGTGAATAAAGTTTTATGTTCCCTGCTCTTAATGTTAAA
Human GFAP NM_001242376.3 (target region for amiR-GFAP-1 underlined;
target region for amiR-GFAP-2 in bold)
(SEQ ID NO: 8)
AGAGCCAGAGCAGGATGGAGAGGAGACGCATCACCTCCGCTGCTCGCCGCTCCTACGTCTCCTC
AGGGGAGATGATGGTGGGGGGCCTGGCTCCTGGCCGCCGTCTGGGTCCTGGCACCCGCCTCTCC
CTGGCTCGAATGCCCCCTCCACTCCCGACCCGGGTGGATTTCTCCCTGGCTGGGGCACTCAATG
CTGGCTTCAAGGAGACCCGGGCCAGTGAGCGGGCAGAGATGATGGAGCTCAATGACCGCTTTGC
CAGCTACATCGAGAAGGTTCGCTTCCTGGAACAGCAAAACAAGGCGCTGGCTGCTGAGCTGAAC
CAGCTGCGGGCCAAGGAGCCCACCAAGCTGGCAGACGTCTACCAGGCTGAGCTGCGAGAGCTGC
GGCTGCGGCTCGATCAACTCACCGCCAACAGCGCCCGGCTGGAGGTTGAGAGGGACAATCTGGC
ACAGGACCTGGCCACTGTGAGGCAGAAGCTCCAGGATGAAACCAACCTGAGGCTGGAAGCCGAG
AACAACCTGGCTGCCTATAGACAGGAAGCAGATGAAGCCACCCTGGCCCGTCTGGATCTGGAGA
GGAAGATTGAGTCGCTGGAGGAGGAGATCCGGTTCTTGAGGAAGATCCACGAGGAGGAGGTTCG
GGAACTCCAGGAGCAGCTGGCCCGACAGCAGGTCCATGTGGAGCTTGACGTGGCCAAGCCAGAC
CTCACCGCAGCCCTGAAAGAGATCCGCACGCAGTATGAGGCAATGGCGTCCAGCAACATGCATG
AAGCCGAAGAGTGGTACCGCTCCAAGTTTGCAGACCTGACAGACGCTGCTGCCCGCAACGCGGA
GCTGCTCCGCCAGGCCAAGCACGAAGCCAACGACTACCGGCGCCAGTTGCAGTCCTTGACCTGC
GACCTGGAGTCTCTGCGCGGCACGAACGAGTCCCTGGAGAGGCAGATGCGCGAGCAGGAGGAGC
GGCACGTGCGGGAGGCGGCCAGTTATCAGGAGGCGCTGGCGCGGCTGGAGGAAGAGGGGCAGAG
CCTCAAGGACGAGATGGCCCGCCACTTGCAGGAGTACCAGGACCTGCTCAATGTCAAGCTGGCC
CTGGACATCGAGATCGCCACCTACAGGAAGCTGCTAGAGGGCGAGGAGAACCGGATCACCATTC
CCGTGCAGACCTTCTCCAACCTGCAGATTCGAGGTCAGTACAGCAGGGCCTCGTGGGAAGGGCA
CTGGAGTCCTGCCCCCTCCTCCAGGGCCTGTAGGTTGCTCCAGACTGGGACTGAGGATCAGGGC
AAAGGGATCCAGCTCTCCCTGGGGGCCTTCGTGACACTGCAGCGCTCCTAGCCAGAGCCTATCA
TACCAGGGTACTTCTAGGTGGGGCTTGCAGCTGCCCCTGTCCTGCTAGGCCCTGGTCCCTCTTC
CCCTCCCTGCACCCCATTCGACAGCAGAACTGGGTGAGAGCTTGACATCTGCCCTGTCTGCAGA
TCCCTGAGCAAGCACTGCCCTTCTGAGTGTTTTCTGTTTTTGTTTTTTTAACTGCTTGTCACTA
CAGGGGGCAAAAGCACCAAAGACGGGGAAAATCACAAGGTCACAAGATATCTCAAAAGCCTCAC
AATACGAGTTATACCAATACAGGCTCACCAGATTGTAAATGGAACGCCGCCGGCTCGCGGTTAG
CTGCCTGCCTCTCAGACACGGCGCTTTGCCCAGCTTGACAGGGAGTGAGCCTCACCCACCCCAT
CCTCCCAATCCCCCTGAGTTCCCTCTTCCCAGGCTTCCCCTAAAGGGCCTGGACTGCGTCATTT
TCCCAGGAACTGCAGTGCCCAGCCCAGGACGTGGTACAGAGTAACTGTACATTAAACTGGCAGA
GCTTGTTAGTGGTAAAGGTGGTGAGTCCTTGGGTGCGCAGTGGAGCTGCTCTGGGGCCTCTGAG
CAAGCAGCAGCCTCTGTCTCACCTCTTCCTGTCACTGGGAGGGCCCCTTGGGTCTCGCTGTGCC
TGGACGCCAGGCTCTCTGCTTTATTCTTTCATCCCTGAGGCTCCATCGCTCAGCTCAGTGCTGA
CTCAGTTCAGAGGATTCTTCCCTCAGGACCGCAGCTCTTGCAGTGAATAAAGTTTTATGTTCCC
TGCTCTTAATGTTAAA
Human GFAP NM_001363846.2 (target region for amiR-GFAP-1 underlined;
target region for amiR-GFAP-2 in bold)
(SEQ ID NO: 9)
AGAGCCAGAGCAGGATGGAGAGGAGACGCATCACCTCCGCTGCTCGCCGCTCCTACGTCTCCTCAGGGGA
GATGATGGTGGGGGGCCTGGCTCCTGGCCGCCGTCTGGGTCCTGGCACCCGCCTCTCCCTGGCTCGAATG
CCCCCTCCACTCCCGACCCGGGTGGATTTCTCCCTGGCTGGGGCACTCAATGCTGGCTTCAAGGAGACCC
GGGCCAGTGAGCGGGCAGAGATGATGGAGCTCAATGACCGCTTTGCCAGCTACATCGAGAAGGTTCGCTT
CCTGGAACAGCAAAACAAGGCGCTGGCTGCTGAGCTGAACCAGCTGCGGGCCAAGGAGCCCACCAAGCTG
GCAGACGTCTACCAGGCTGAGCTGCGAGAGCTGCGGCTGCGGCTCGATCAACTCACCGCCAACAGCGCCC
GGCTGGAGGTTGAGAGGGACAATCTGGCACAGGACCTGGCCACTGTGAGGCAGAAGCTCCAGGATGAAAC
CAACCTGAGGCTGGAAGCCGAGAACAACCTGGCTGCCTATAGACAGGAAGCAGATGAAGCCACCCTGGCC
CGTCTGGATCTGGAGAGGAAGATTGAGTCGCTGGAGGAGGAGATCCGGTTCTTGAGGAAGATCCACGAGG
AGGAGGTTCGGGAACTCCAGGAGCAGCTGGCCCGACAGCAGGTCCATGTGGAGCTTGACGTGGCCAAGCC
AGACCTCACCGCAGCCCTGAAAGAGATCCGCACGCAGTATGAGGCAATGGCGTCCAGCAACATGCATGAA
GCCGAAGAGTGGTACCGCTCCAAGTTTGCAGACCTGACAGACGCTGCTGCCCGCAACGCGGAGCTGCTCC
GCCAGGCCAAGCACGAAGCCAACGACTACCGGCGCCAGTTGCAGTCCTTGACCTGCGACCTGGAGTCTCT
GCGCGGCACGAACGAGTCCCTGGAGAGGCAGATGCGCGAGCAGGAGGAGCGGCACGTGCGGGAGGCGGCC
AGTTATCAGGAGGCGCTGGCGCGGCTGGAGGAAGAGGGGCAGAGCCTCAAGGACGAGATGGCCCGCCACT
TGCAGGAGTACCAGGACCTGCTCAATGTCAAGCTGGCCCTGGACATCGAGATCGCCACCTACAGGAAGCT
GCTAGAGGGCGAGGAGAACCGGATCACCATTCCCGTGCAGACCTTCTCCAACCTGCAGATTCGAGGGGGC
AAAAGCACCAAAGACGGGGAAAATCACAAGGTCACAAGATATCTCAAAAGCCTCACAATACGAGTTATAC
CAATACAGGCTCACCAGATTGTAAATGGAACGCCGCCGGCTCGCGAAACCAGCCTGGACACCAAGTCTGT
GTCAGAAGGCCACCTCAAGAGGAACATCGTGGTGAAGACCGTGGAGATGCGGGATGGAGAGGTCATTAAG
GAGTCCAAGCAGGAGCACAAGGATGTGATGTGAGGCAGGACCCACCTGGTGGCCTCTGCCCCGTCTCATG
AGGGGCCCGAGCAGAAGCAGGATAGTTGCTCCGCCTCTGCTGGCACATTTCCCCAGACCTGAGCTCCCCA
CCACCCCAGCTGCTCCCCTCCCTCCTCTGTCCCTAGGTCAGCTTGCTGCCCTAGGCTCCGTCAGTATCAG
GCCTGCCAGACGGCACCCACCCAGCACCCAGCAACTCCAACTAACAAGAAACTCACCCCCAAGGGGCAGT
CTGGAGGGGCATGGCCAGCAGCTTGCGTTAGAATGAGGAGGAAGGAGAGAAGGGGAGGAGGGCGGGGGGC
ACCTACTACATCGCCCTCCACATCCCTGATTCCTGTTGTTATGGAAACTGTTGCCAGAGATGGAGGTTCT
CTCGGAGTATCTGGGAACTGTGCCTTTGAGTTTCCTCAGGCTGCTGGAGGAAAACTGAGACTCAGACAGG
AAAGGGAAGGCCCCACAGACAAGGTAGCCCTGGCCAGAGGCTTGTTTTGTCTTTTGGTTTTTATGAGGTG
GGATATCCCTATGCTGCCTAGGCTGACCTTGAACTCCTGGGCTCAAGCAGTCTACCCACCTCAGCCTCCT
GTGTAGCTGGGATTATAGATTGGAGCCACCATGCCCAGCTCAGAGGGTTGTTCTCCTAGACTGACCCTGA
TCAGTCTAAGATGGGTGGGGACGTCCTGCCACCTGGGGCAGTCACCTGCCCAGATCCCAGAAGGACCTCC
TGAGCGATGACTCAAGTGTCTCAGTCCACCTGAGCTGCCATCCAGGGATGCCATCTGTGGGCACGCTGTG
GGCAGGTGGGAGCTTGATTCTCAGCACTTGGGGGATCTGTTGTGTACGTGGAGAGGGATGAGGTGCTGGG
AGGGATAGAGGGGGGCTGCCTGGCCCCCAGCTGTGGGTACAGAGAGGTCAAGCCCAGGAGGACTGCCCCG
TGCAGACTGGAGGGGACGCTGGTAGAGATGGAGGAGGAGGCAATTGGGATGGCGCTAGGCATACAAGTAG
GGGTTGTGGGTGACCAGTTGCACTTGGCCTCTGGATTGTGGGAATTAAGGAAGTGACTCATCCTCTTGAA
GATGCTGAAACAGGAGAGAAAGGGGATGTATCCATGGGGGCAGGGCATGACTTTGTCCCATTTCTAAAGG
CCTCTTCCTTGCTGTGTCATACCAGGCCGCCCCAGCCTCTGAGCCCCTGGGACTGCTGCTTCTTAACCCC
AGTAAGCCACTGCCACACGTCTGACCCTCTCCACCCCATAGTGACCGGCTGCTTTTCCCTAAGCCAAGGG
CCTCTTGCGGTCCCTTCTTACTCACACACAAAATGTACCCAGTATTCTAGGTAGTGCCCTATTTTACAAT
TGTAAAACTGAGGCACGAGCAAAGTGAAGACACTGGCTCATATTCCTGCAGCCTGGAGGCCGGGTGCTCA
GGGCTGACACGTCCACCCCAGTGCACCCACTCTGCTTTGACTGAGCAGACTGGTGAGCAGACTGGTGGGA
TCTGTGCCCAGAGATGGGACTGGGAGGGCCCACTTCAGGGTTCTCCTCTCCCCTCTAAGGCCGAAGAAGG
GTCCTTCCCTCTCCCCAAGACTTGGTGTCCTTTCCCTCCACTCCTTCCTGCCACCTGCTGCTGCTGCTGC
TGCTAATCTTCAGGGCACTGCTGCTGCCTTTAGTCGCTGAGGAAAAATAAAGACAAATGCTGCGCCCTTC
CCCA
Mouse GFAP NM_001131020.1 (target region for amiR-GFAP-1 underlined;
target region for amiR-GFAP-2 in bold)
(SEQ ID NO: 10)
CCAGGAAGTCAGGGGCAGATTTAGTCCAACCCGTTCCTCCATAAAGGCCCTGACATCCCAGGAG
CCAGCAGAGGCAGGGCAGGATGGAGCGGAGACGCATCACCTCTGCGCGCCGCTCCTATGCCTCC
GAGACGGTGGTCAGGGGCCTCGGTCCTAGTCGACAACTGGGTACCATGCCACGCTTCTCCTTGT
CTCGAATGACTCCTCCACTCCCTGCCAGGGTGGACTTCTCCCTGGCCGGGGCGCTCAATGCTGG
CTTCAAGGAGACACGGGCGAGCGAGCGTGCAGAGATGATGGAGCTCAATGACCGCTTTGCTAGC
TACATCGAGAAGGTCCGCTTCCTGGAACAGCAAAACAAGGCGCTGGCAGCTGAACTGAACCAGC
TTCGAGCCAAGGAGCCCACCAAACTGGCTGATGTCTACCAGGCGGAGCTTCGGGAGCTGCGGCT
GCGGCTGGACCAGCTTACGGCCAACAGTGCCCGGCTGGAGGTGGAGAGGGACAACTTTGCACAG
GACCTCGGCACCCTGAGGCAGAAGCTCCAAGATGAAACCAACCTGAGGCTGGAGGCAGAGAACA
ACCTGGCTGCGTATAGACAGGAGGCAGATGAAGCCACCCTGGCTCGTGTGGATTTGGAGAGAAA
GGTTGAATCGCTGGAGGAGGAGATCCAGTTCTTAAGGAAGATCTATGAGGAGGAAGTTCGAGAA
CTCCGGGAGCAGCTGGCCCAACAGCAGGTCCACGTGGAGATGGATGTGGCCAAGCCAGACCTCA
CAGCGGCCCTGAGAGAGATTCGCACTCAATACGAGGCAGTGGCCACCAGTAACATGCAAGAGAC
AGAGGAGTGGTATCGGTCTAAGTTTGCAGACCTCACAGACGCTGCGTCCCGCAACGCAGAGCTG
CTCCGCCAAGCCAAGCACGAAGCTAACGACTATCGCCGCCAACTGCAGGCCTTGACCTGCGATC
TGGAGTCCCTGCGCGGCACGAACGAGTCCCTAGAGCGGCAAATGCGCGAACAGGAAGAGCGCCA
TGCGCGGGAGTCGGCCAGTTACCAGGAGGCACTTGCTCGGCTGGAGGAGGAGGGCCAAAGCCTC
AAGGAGGAGATGGCCCGCCACCTGCAGGAGTACCAGGATCTACTCAACGTTAAGCTAGCCCTGG
ACATCGAGATCGCCACCTACAGGAAATTGCTGGAGGGCGAAGAAAACCGCATCACCATTCCTGT
ACAGACTTTCTCCAACCTCCAGATCCGAGGGGGCAAAAGCACCAAAGAAGGGGAAGGCCAAAAA
GTCACAAGACCTCTCAAAAGGCTCACAATACAAGTTGTCCCAATACAGGCTCACCAGATTGAAA
ATGGAGCCCTGCCAGCTCTCCCTTAGATAGATGCGTGCTCCAGCTCTCCCTTAGATAGAGGCGT
GCTCCAGCTCTCCCTTAGATAGAGGCGTGCTCCAGCTCTCCCTTAGATAGAGGCGTGCTCCAGC
TCTCCCTTAGATAGAGGCGTGCTCCAGCTCTCCCTTAGATAGAGGCGTGCTCCAGCTCTCCCTT
AGATAGATGCGCGCATTTCAGCCACACCTTTCCAGCTTGTCTTCTTCCTCCCAGGCCTCCTCTA
AGGGACTGAACCATGTCCTTTGTCTAGAAGCTTCCAGGCCCACCCTAGGTCCTGGCTCTGTGTA
ATTAGGTTATACCGATAGAGCTAGCCTATGCTAAAGGTTAGGTTGTACTAACAGAGCTAGCCTA
TGCTAAAGGTTAGGTTGTACTAATAGAGCTAGTCTTATGCTAAAGGTTAGGTTGTATTAACAGA
GCTAGCCTATGCTAAAGGTTAGGTTGTACTAACAGAGCTAGTCATGTTAAGTTAGGTTGTACTA
ACAGAGCTAGCTTATGCTAAAGTTAGGTTGTACTAACATAGCTAGCCTATGCTAAAGGTTTGGT
TGTACTAACAGAGCTGGCCTATGTTAAAGGTTAGGTTGTACTAATAGAGCTAGTCTTATGCTAA
AGGTTAGGTTGTATTAACAGAGCTAACCTATGCTAAAGGTTAGGTTGTACTAACAGAGCTGGCC
TATGTTAAAGGTTAGGTTGTACTAATAGAGCTAGCCTATGCTAAAGGTTAGGTTGTACTAACAG
AGCTGGCCTATGTTAAAGGTTAGGTTTTACTAATAGAGCTAGTCTTATGCTAAAGGTTAGGTTG
TACTAACAGAGCGAGCCTATGCTAAAGATTAGGTTATATTAACAGAGCTAGCCTATGCTAAAGG
TTAGGTTGTACTAATAGAGCTAGCCTATGCTAAAGGTTAGGTTGTACTAATAGAGCTAGCCTAT
GCTAAAGGTTAGGTTGTATTAACAGAGCTAGCCAATGTTAAAGGCAGCAAGTCCCTGGGAGCTC
CAAGGAGATACTCTGAACCCTCTGAGCAAATGCCTCCGGCTCACCAGTTTCTGTCGCTAGGTGG
TCCCCTTGGGTCTTGCAGTGCCTGTGGGCAGGCTCTGTGTTTGATTCATGTGTCCCCAGAGTTC
TATTGCTTCACTTCAGTGCTGATTCAGCCCAGAGGGTTAGTTAGTTCCCTCTGGACGGCTGCTC
TTGTAGTGAATAAAGCTTTATGCTCCCTGCTCTTCATTTT
Mouse GFAP NM_010277.3 (target region for amiR-GFAP-1 underlined;
target region for amiR-GFAP-2 in bold)
(SEQ ID NO: 11)
CCAGGAAGTCAGGGGCAGATTTAGTCCAACCCGTTCCTCCATAAAGGCCCTGACATCCCAGGAG
CCAGCAGAGGCAGGGCAGGATGGAGCGGAGACGCATCACCTCTGCGCGCCGCTCCTATGCCTCC
GAGACGGTGGTCAGGGGCCTCGGTCCTAGTCGACAACTGGGTACCATGCCACGCTTCTCCTTGT
CTCGAATGACTCCTCCACTCCCTGCCAGGGTGGACTTCTCCCTGGCCGGGGCGCTCAATGCTGG
CTTCAAGGAGACACGGGCGAGCGAGCGTGCAGAGATGATGGAGCTCAATGACCGCTTTGCTAGC
TACATCGAGAAGGTCCGCTTCCTGGAACAGCAAAACAAGGCGCTGGCAGCTGAACTGAACCAGC
TTCGAGCCAAGGAGCCCACCAAACTGGCTGATGTCTACCAGGCGGAGCTTCGGGAGCTGCGGCT
GCGGCTGGACCAGCTTACGGCCAACAGTGCCCGGCTGGAGGTGGAGAGGGACAACTTTGCACAG
GACCTCGGCACCCTGAGGCAGAAGCTCCAAGATGAAACCAACCTGAGGCTGGAGGCAGAGAACA
ACCTGGCTGCGTATAGACAGGAGGCAGATGAAGCCACCCTGGCTCGTGTGGATTTGGAGAGAAA
GGTTGAATCGCTGGAGGAGGAGATCCAGTTCTTAAGGAAGATCTATGAGGAGGAAGTTCGAGAA
CTCCGGGAGCAGCTGGCCCAACAGCAGGTCCACGTGGAGATGGATGTGGCCAAGCCAGACCTCA
CAGCGGCCCTGAGAGAGATTCGCACTCAATACGAGGCAGTGGCCACCAGTAACATGCAAGAGAC
AGAGGAGTGGTATCGGTCTAAGTTTGCAGACCTCACAGACGCTGCGTCCCGCAACGCAGAGCTG
CTCCGCCAAGCCAAGCACGAAGCTAACGACTATCGCCGCCAACTGCAGGCCTTGACCTGCGATC
TGGAGTCCCTGCGCGGCACGAACGAGTCCCTAGAGCGGCAAATGCGCGAACAGGAAGAGCGCCA
TGCGCGGGAGTCGGCCAGTTACCAGGAGGCACTTGCTCGGCTGGAGGAGGAGGGCCAAAGCCTC
AAGGAGGAGATGGCCCGCCACCTGCAGGAGTACCAGGATCTACTCAACGTTAAGCTAGCCCTGG
ACATCGAGATCGCCACCTACAGGAAATTGCTGGAGGGCGAAGAAAACCGCATCACCATTCCTGT
ACAGACTTTCTCCAACCTCCAGATCCGAGAAACCAGCCTGGACACCAAATCCGTGTCAGAAGGC
CACCTCAAGAGGAACATCGTGGTAAAGACTGTGGAGATGCGGGATGGTGAGGTCATTAAGGACT
CGAAGCAGGAGCACAAGGACGTGGTGATGTGAGGTGTGCCCACCTGGTGGCCCTTGCCATGCAG
TGTGAGGGCCCAAAGCTTATCCTCAAATAGTCCTGTTTGCCAGGCTCAGTTCCCACCCACACCA
GCACTTCCCTTCCTTCTGGTTTTCTGCCTGTGTGCTGCCCAAGGCTCAATCAGTGCTAAGCTTC
ATAGATGGCATATACCCTTCACCTTCAACTAACAGGATACTCACCCCAAAGGCGCAGTCAGGAG
GGGAGGGAACCCCAGCTGGGTTAGAATTGGAAGGGAAGAGGAAAGATGAGCAGAGTAGAGAGAT
TTAACAAATCACTTCCTTCATCCTTGTTGTTATGGAAACCGTTGCCAGAGCTGGAAGTTTCCAC
AGGCTGCTGGAGCTAGACAACAATTCAGACAGAAAGGGAAAGTCCCTGAGGCAAAGTCTCTCTA
GCCAGAGACCTATGCATCCCGAATGGCCACTAAGGCAGTCCTGAAGGGCCCTCCAGGGTGATGA
CTCCAGTGTGTCAGCCCCACTGAGCAGCTATGCAGGTTGACTGCCCACAGGCATGTGGAAACTT
GGTTCTCAGCACTTGGCAGGATCTATGGCATAAGTGGAGAGGGAAGGTGTACTGGACGGCGGAG
AGGAGGGCTCCCTGGCCCCTAAGTGTGGATGCAGAGAGGTGGAGCCCAGGAAGGGTCTCTGCTT
AGGCTGCAGGGGTGCCAATGGCAGAGGCACTGGTAGAGATCATTTGGACACTGGAGTTGAAAGT
TACAGGCAATCTGTTACACTTGGCTCTGAATCCTATGAATCAAGGAAATAACCCGTTCTCTGGA
AGACACTGAAACAGGAGAGAGGGACTTCCGTCCACTGGGCAGGGTACAGATGTGTCTCAGTTGT
GAAGGTCTATTCCTGGCTGCACAGTCCCCATCCGCTCAGTCATCTTACCCTGTGACTGCTCTCA
GCCCTGAAGAATCCACAACCATCCTTCCAAGGTTCTCCATCCCCACAATGACTAGCTGTTGCTC
TCCAAGCTAAGGGACCATTCCCTGTCTTATGCATATACGTAATGTCACCTATTTAGGTATCATC
CTATTTGAGAGTTTGAGGAACTGAAACGTGTTGTGTTCAAGCAGCCTGGTGGCTAGTGCCTTCA
TATTAGAGCACCTTCTCTGAGGCTGATTGGTGGGCAGGTAGGGAAGACATTGAGCAGACAGTGT
CCGCTCAGTTGTCCTTCCCTCCCTTCCAAGGTCCCTCCCTCTTTCCAGGACATCGCCCCCCCAC
CCCACCCCTCCTTTCCACCTCCGCTAACCTCCAGAGCAGTACTGTCACCTTTACTCACTGGGCA
GAAATAAAGACATGTGCCATAGACTTCCAAAAAAAAAAAAAAAAA
amiR-GFAP-2 Guide Strand
(SEQ ID NO: 12)
TCATTGAGCTCCATCATCTCT
amiR-GFAP-2 Passenger Strand
(SEQ ID NO: 13)
AGAGATGAACGGGCTCAATGA
amiR-GFAP-1 Guide Strand
(SEQ ID NO: 14)
TTGTTTTGCTGTTCCAGGAAG
amiR-GFAP-1 Passenger Strand
(SEQ ID NO: 15)
CTTCCTGGTTCGGCAAAACAA
amiR-GFAP-2 target sequence
(SEQ ID NO: 16)
AGAGATGATGGAGCTCAATGA
amiR-GFAP-1 target sequence
(SEQ ID NO: 17)
CTTCCTGGAACAGCAAAACAA

What is claimed is:

1. An isolated nucleic acid comprising a nucleic acid sequence encoding an artificial microRNA (amiRNA) that targets a glial fibrillary acidic protein (GFAP) RNA transcript, wherein the nucleic acid sequence is flanked by adeno-associated virus inverted terminal repeats (ITRs).

2. The isolated nucleic acid of claim 1, wherein the amiRNA comprises:
(i) a nucleic acid sequence encoding a pri-miRNA scaffold;
(ii) a nucleic acid sequence encoding a guide strand; and,
(iii) a nucleic acid sequence encoding a passenger strand,
wherein, the pri-miRNA scaffold is derived from a naturally-occurring pri-miRNA and comprises at least one flanking sequence and a loop-forming sequence comprising at least 4 nucleotides.

3. The isolated nucleic acid of claim 1 or claim 2, wherein the pri-miRNA scaffold is derived from a pri-miRNA selected from the group consisting of pri-MIR-21, pri-MIR-22,pri-MIR-26a, pri-MIR-30a, pri-MIR-33, pri-MIR-122, pri-MIR-375, pri-MIR-199, pri-MIR-99,pri-MIR-194, pri-MIR-155, and pri-MIR-451.

4. The isolated nucleic acid of any one of claims 1 to 3, wherein the nucleic acid sequence encoding the guide strand and/or the passenger strand comprises a sequence that is at least 70%, 75%, 80%, 85%, 90%, 95%, or 99% identical to the nucleotide sequence set forth in SEQ ID NO: 1 or 2.

5. The isolated nucleic acid of any one of claims 1 to 3, wherein the nucleic acid sequence encoding the guide strand and/or the passenger strand comprises the sequence set forth in SEQ ID NO: 1 or 2.

6. The isolated nucleic acid of any one of claims 1 to 5 further comprising a promoter operably linked to the nucleic acid sequence encoding the amiRNA.

7. The isolated nucleic acid of claim 6, wherein the promoter comprises a chicken beta actin (CB) promoter or a GFAP promoter.

8. The isolated nucleic acid of claim 7, wherein the endogenous GFAP promoter is a GfaABC1D promoter, optionally wherein the GfaABC1D promoter comprises the sequence set forth in SEQ ID NO: 3.

9. The isolated nucleic acid of any one of claims 1 to 8, wherein the nucleic acid comprises a self-complementary AAV (scAAV) vector.

10. The isolated nucleic acid of any one of claims 1 to 9 comprising the sequence set forth in SEQ ID NO: 4 or 5.

11. A recombinant adeno-associated virus (rAAV) comprising:
(i) the isolated nucleic acid of any one of claims 1 to 10; and
(ii) at least one AAV capsid protein.

12. The rAAV of claim 11, wherein the at least one capsid protein has a serotype selected from an AAV2, AAV3, AAV4, AAV5, AAV6, AAV7, AAV8, AAVrh8, AAV9, AAV10, or AAVrh10 capsid protein.

13. The rAAV of claim 11 or 12, wherein the at least one capsid protein is an AAV9 capsid protein.

14. The rAAV of any one of claims 11 to 13, wherein the rAAV is a self-complementary AAV (scAAV).

15. A method for reducing glial fibrillary acidic protein (GFAP) in a cell or subject, the method comprising administering the isolated nucleic acid of any one of claims 1 to 10 or the rAAV of any one of claims 11 to 14 to the cell or subject.

16. The method of claim 15, wherein GFAP is reduced in the brain.

17. The method of claim 16, wherein GFAP is reduced in the hippocampus and/or olfactory bulbs.

18. The method of claim 15, wherein the cell or subject is a mammalian cell or mammalian subject.

19. The method of any one of claims 15 to 18, wherein the cell or subject is a mouse, rat, or human cell or subject.

20. The method of any one of claims 15 to 19, wherein the cell or subject comprises one or more mutations in a GFAP gene, optionally wherein the one or more mutations comprise heterozygous mutations in each copy of a GFAP gene.

21. The method of any one of claims 15 to 20, wherein the cell or subject has or is suspected of having Alexander disease (AxD).

22. A method for reducing Rosenthal fiber formation in a subject, the method comprising administering the isolated nucleic acid of any one of claims 1 to 10 or the rAAV of any one of claims 11 to 14 to the cell or subject.

23. The method of claim 22, wherein Rosenthal fiber formation in a subject is reduced in the brain.

24. The method of claim 23, wherein Rosenthal fiber formation in a subject is reduced in the hippocampus and/or olfactory bulbs.

25. The method of claim 22, wherein the subject is mammal.

26. The method of any one of claims 22 to 25, wherein the subject is a mouse, rat, or human.

27. The method of any one of claims 22 to 26, wherein the subject comprises one or more mutations in a GFAP gene, optionally wherein the one or more mutations comprise heterozygous mutations in each copy of a GFAP gene.

28. The method of any one of claims 22 to 27, wherein the subject has or is suspected of having Alexander disease (AxD).

29. A method for treating Alexander disease (AxD) in a subject, the method comprising administering to the subject the isolated nucleic acid of any one of claims 1 to 10 or the rAAV of any one of claims 11 to 14.

30. The method of claim 29, wherein the subject is a mammal.

31. The method of claim 29 or 30, wherein the subject is a mouse, rat, or human.

32. The method of any one of claims 29 to 31, wherein the subject comprises one or more mutations in a GFAP gene, optionally wherein the one or more mutations comprise heterozygous mutations in each copy of a GFAP gene.

33. The method of any one of claims 29 to 32, wherein the administration comprises systemic administration, optionally wherein the systemic administration comprises intravenous injection.

34. The method of any one of claims 29 to 33, wherein the administration results in reduced Rosenthal fiber formation in the subject.
